(19) United States
(12) Patent Application Publication
(10) Pub. No.: US 2019/0330659 A1
(43) Pub. Date: Oct. 31, 2019

(54) SCARLESS DNA ASSEMBLY AND GENOME EDITING USING CRISPR/CPF1 AND DNA LIGASE

(71) Applicant: Zymergen Inc., Emeryville, CA (US)

(72) Inventors: William C. DeLoache, Oakland, CA (US); Hendrik Marinus van Rossum, Oakland, CA (US); Kedar Gautam Patel, Fremont, CA (US)

(21) Appl. No.: 16/310,895

(22) PCT Filed: Jul. 14, 2017
(86) PCT No.: PCT/US2017/042245

§ 371 Date: Dec. 18, 2018

Related U.S. Application Data
(60) Provisional application No. 62/362,909, filed on Jul. 15, 2016.

(51) Int. Cl. C12N 15/90; C12N 15/66; C12N 15/113; C12N 15/10; C12N 9/00

(52) U.S. Cl. CPC C12N 15/902; C12N 15/66; C12N 15/113; C12Q 2521/301; C12N 9/93; C12N 2310/20; C12N 15/1031

(57) ABSTRACT

The disclosure describes a scarless DNA assembly and genome editing methodology termed “CLIC” (CRISPR and Ligase Cloning), which utilizes a CRISPR/Cpf1 complex and DNA ligase to perform programmable gene editing and nucleotide assembly. The CLIC process is highly amenable to applications in vitro for the scarless assembly of a plurality of DNA parts simultaneously or in vivo for the site-specific insertion of one or more DNA molecules into the host genome.

### CROSS-REFERENCE TO RELATED APPLICATIONS

This application claims the benefit of priority to U.S. provisional application No. 62/362,909 filed on Jul., 15 2016, which is hereby incorporated by reference in its entirety, including all descriptions, references, figures, and claims for all purposes.

### FIELD

The present disclosure generally relates to systems, methods, and compositions used for guided genetic sequence editing in vivo and in vitro. The disclosure describes, inter alia, methods of using guided sequence editing complexes for improved DNA cloning, assembly of oligonucleotides, and for the improvement of microorganisms.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: ZYMR_002_01WO_SeqList_ST25.txt, date recorded: Jul. 14, 2017; file size: 797 kilobytes).

### BACKGROUND

A major area of interest in biology is the in vivo and in vitro targeted editing of genetic sequences. Clustered regularly interspaced short palindromic repeats (CRISPR) systems are a new class of genome-editing tools capable of targeting and modifying selected target DNA loci.

CRISPR editing begins with a double stranded DNA break catalyzed by the CRISPR complex that triggers a cell's homology-directed repair (HDR) mechanisms. Modern gene editing techniques exploit the HDR process to knock in replacement DNA sections with desired sequence modifications.

Unfortunately, the success rate of HDR from traditional CRISPR systems remains extremely low. Moreover, HDR failures often result in non-homologous end-joining at the site of the DNA break, which can inadvertently result in frameshift mutations, and loss of function of the targeted allele. Finally, CRISPR editing function requires the presence of homologous recombination machinery that is not available for conducting in vitro cloning reactions, or in vivo reactions in organisms lacking homologous recombination genes.

Thus, there is a need for improved compositions and methods for targeted alteration of genetic sequences.

### SUMMARY OF THE DISCLOSURE

In some embodiments, the present disclosure teaches methods, compositions, and kits for scarless “single pot” in vivo and in vitro DNA assembly reactions. Thus in some embodiments, the present disclosure teaches methods of digesting DNA with endonucleases. In some embodiments, the present disclosure teaches digesting DNA with CRISPR endonucleases. In some embodiments, the present disclosure teaches digesting DNA with Type V-class 2 CRISPR endonucleases. In some embodiments, the present disclosure teaches digesting DNA with Cpf1 endonucleases.

In some embodiments, the present disclosure teaches a CRISPR and Ligase Cloning method (termed “CLIC”). In some embodiments, the present disclosure teaches that CLIC is a method for DNA assembly that relies on the CRISPR nuclease Cpf1 to digest DNA molecules, leaving behind three to five base-pair sticky ends whose sequence can be controlled through the design of crRNA guide sequences (e.g., by designing the location of the Cpf1 cut). In some embodiments, these sticky ends are then annealed and ligated together with a DNA ligase in order to join two or more digested fragments into a fully assembled construct or genome without the addition of any genetic scars.

In some embodiments, the present disclosure teaches “single pot” one-reaction DNA assembly reactions that do not require inactivation of the endonuclease. In some embodiments, the methods of the present disclosure can be applied to multi-fragment assembly reactions. In some embodiments, the CLIC methods of the present disclosure capitalizes on the properties of class 2 CRISPR endonucleases, which cleave DNA at a location outside of their binding site. Thus, in some embodiments, the present disclosure teaches targeting class 2 CRISPR endonuclease target sites to locations of DNA that will be removed during the DNA assembly process, such that digested DNA regions cease to be substrates for the endonuclease. The present disclosure teaches that digested DNA fragments of the present invention can therefore be annealed and ligated to other DNA fragments in the same reaction as the CRISPR class 2 endonuclease cutting.

For example, in some embodiments, the present disclosure teaches a method for assembling gene constructs in vitro from a plurality of DNA fragments, said method comprising the steps of: (a) providing a plurality of DNA fragments comprising a first and second DNA fragment, wherein said first DNA fragment comprises a sequence overlap of at least three nucleic acids anywhere within the second DNA fragment; (b) digesting the first DNA fragment with a Cpf1 CRISPR system, thereby creating a sticky DNA end at the 5′ and/or 3′ of said first DNA fragment, wherein said digested first DNA fragment ceases to be a target for said Cpf1 CRISPR system; (c) annealing the sticky end of the digested first DNA fragment from step (b) to a second compatible sticky end on the second DNA fragment; and (d) ligating the annealed DNA fragments from step (c) together; wherein the resulting annealed product is an assembled construct.

The methods of the present disclosure are in some embodiments not limited to the assembly of only two DNA fragments. The present disclosure teaches methods for assembling multiple fragments. The methods of the present disclosure also provide users control of the order and directionality in which fragments are assembled. In some embodiments, the present disclosure teaches that the sticky ends created by the endonuclease digestions can be targeted to regions to create sticky ends that are only compatible when combined in a selected order and direction. See FIG. 5 for an illustration of one such embodiment of the present disclosure.

In some embodiments, the present disclosure teaches the use of crRNA with programmable guide sequences, which allow users to target to any sequence in the proximity of a compatible PAM. Thus the methods of the present invention, in some embodiments, do not require the introduction of restriction enzymes binding sites into DNA assembly reactions.

Thus in some embodiments, the present disclosure teaches a method of for assembling gene constructs, wherein no genetic scars are introduced into the assembled construct from practicing the method.

In some embodiments, the Cpf1 CRISPR systems of the present disclosure comprise i) a Cpf1 endonuclease, and ii) a crRNA capable of directing sequence-specific binding of the Cpf1 endonuclease to the first DNA fragment.

In other embodiments, the present disclosure teaches methods of expressing the components of Cpf1 CRISPR systems in vivo and in vitro. For example, in some embodiments, the present disclosure teaches cell-free expression systems for Cpf1 endonucleases from encoding polynucleotides. In other embodiments, the present disclosure teaches cell-free transcription, such as commercial DNA-dependent RNA polymerases for the production of crRNAs.

In some embodiments, the Cpf1 endonucleases of the present disclosure are naturally occurring (e.g., they are encoded by polynucleotides found in wild type organisms). In other embodiments, the Cpf1 endonucleases of the present disclosure are non-naturally occurring.

For example, in some embodiments, the present disclosure teaches codon-optimized Cpf1 endonucleases. In other embodiments, the present disclosure teaches engineered Cpf1 endonucleases. Thus in some embodiments, the present disclosure teach Cpf1 endonucleases with Nuclear Localization Signals. In some embodiments, the present disclosure teaches Cpf1 endonucleases with altered sequence for improved activity (e.g., improved kinetics, stability, half-life, compatibility with different PAMs, or functionality in different buffers).

In some embodiments, the present disclosure teaches the use of naturally occurring crRNA sequences (e.g., they are encoded by polynucleotides found in wild type organisms). In other embodiments, the crRNA sequences of the present disclosure are non-naturally occurring. In some embodiments, the crRNAs are engineered to target selected DNA sequences.

In some embodiments, the present disclosure teaches DNA assemblies wherein the Cpf1 CRISPR system of step (b) is targeted to a portion of the first DNA fragment that will be cleaved away from the first DNA fragment, such that the Cpf1 CRISPR system no longer targets the digested first DNA fragment.

In some embodiments, the present disclosure teaches methods of targeting Cpf1 CRISPR systems to cleave assembly DNA fragments in locations that will result in the creation of a sticky end that is compatible with a second DNA fragment (e.g., wherein the endonuclease creates a sticky end corresponding to the sequence overlap between the first DNA fragment and the second DNA fragment, such that the resulting sticky ends can hybridize). Thus, for the purposes of this disclosure, sequence overlap refers to a sequence present anywhere in both of the referenced DNA fragments. For example, a first DNA fragment might contain the sequence AAG at its 5′ end, while the second DNA fragment might contain the same AAG sequence near its center, starting at base pair 200 from its 5′ end.

In some embodiments, the present CLIC reactions are “single pot” such that steps (b) and (d) corresponding to the endonuclease digestion and ligation are conducted in the same reaction without needing to inactivate the Cpf1 CRISPR system, or otherwise purify the sequences between steps of the reaction.

In some embodiments, the present disclosure teaches that one or more DNA fragments in the CLIC reaction can comprise preexisting sticky ends compatible with the sticky end of the digested DNA fragments. For example, the present disclosure includes CLIC reactions in which a circular plasmid is cleaved with a Cpf1 endonuclease to remove an MCS site, which is then ligated to an insertion GOI that either had preexisting sticky ends, or was also digested by the Cpf1 endonuclease.

In some embodiments, the present disclosure teaches that a preexisting sticky end can be created by the staggered hybridization of two oligos with overhangs, or ends created through exonuclease reactions, or prior restriction digestions.

In other embodiments, the present disclosure teaches methods in which step (b) Cpf1 endonuclease digestion further comprises digesting the second DNA fragment with a second Cpf1 CRISPR system, thereby creating a sticky DNA end at the 5′ and/or 3′ of said second DNA fragment, wherein said digested second DNA fragment ceases to be a target for said second Cpf1 CRISPR endonuclease system. See FIG. 2 for an illustration of one such embodiment of the present disclosure.

In some embodiments, the present disclosure teaches that the first Cpf1 CRISPR system and the second Cpf1 CRISPR system are identical, such that a single Cpf1 CRISPR system could be programmed to cleave two or more DNA fragments. This approach is particularly feasible in embodiments in which the second DNA fragment is designed to match the target sequence of the first DNA sequence (e.g., engineering the ends of a gene insert to match the target sequence located on the inner edges of the MCS of the destination plasmid). In some embodiments, using the same Cpf1 CRISPR can still produce different sticky ends to maintain control over assembly order and direction.

In some embodiments, the present disclosure also teaches a method for editing the genome of a cell in vivo, said method comprising the steps of: a) introducing into the cell a Cpf1 CRISPR system comprising one or more vectors comprising: i) a first polynucleotide encoding a first crRNA that hybridizes to a first selected target sequence within the genome of the cell; ii) a second polynucleotide encoding a second crRNA that hybridizes to a second selected target within the genome of the cell; and ii) a third polynucleotide encoding a Cpf1 endonuclease; wherein components (a), (b), and (c) are expressed in the cell, and the Cpf1 endonuclease cleaves the cell's genome at the selected target sequences, thereby producing sticky ends on the cleaved ends of the cell's genome; wherein the first and second target sequences are positioned in an outwardly facing inverse orientation of a portion of the cell's genome slated for removal, such that removal of said portion of the cell's genome will also remove the first and second target sites from the genome; (b) annealing the resulting genome sticky ends to each other; and (c) ligating the annealed genome sticky ends from step (b).

Thus in some embodiments, the present disclosure teaches methods of introducing Cpf1 CRISPR complexes into cells by introducing polynucleotides capable of expressing the necessary crRNA and Cpf1 endonuclease components.

In some embodiments, the present disclosure also teaches methods of introducing insert sequences into cells via transformation. In some embodiments, the present disclosure teaches transformation of inserts sequences with preexisting sticky ends. In other embodiments, the present disclosure teaches insertion of sequences that will be processed in vivo. In some embodiments, the insert sequences of the present disclosure are introduced into the cell in linear form. In other embodiments, the sequences of the present disclosure are introduced in a circular plasmid. In some embodiments, the present disclosure teaches that the circular plasmid will be a replicating plasmid. In some embodiments the introduction of each Cpf1 CRISPR system component can be done in parallel (e.g., multiple plasmids with all the pieces), or sequentially (e.g., introducing some components first, then other components).

In some embodiments, the present disclosure also teaches methods of integrating selected components of the Cpf1 CRISPR system into the genome of the cell that will be edited. For example, in some embodiments, the cell may already comprise a polynucleotide encoding the Cpf1 endonuclease. In other embodiments, the cell may already comprise a polynucleotide encoding for a ligase.

Thus, in some embodiments, the present disclosure teaches that the one or more vectors of step (a) of the in vivo CLIC method may also comprise a fourth insert polynucleotide, wherein said insert polynucleotide is also cleaved by the Cpf1 endonuclease, thereby creating sticky ends on the insert polynucleotide that are compatible with the sticky ends of the cell's genome; wherein the annealing step (b) is modified to anneal the sticky ends of the genome to the sticky ends of the insert polynucleotide; and wherein the ligating step (c) is modified to ligate the annealed genome and insert sticky ends.

The present disclosure also teaches embodiments of the in vivo CLIC gene editing methods that do not introduce any genetic scars.

In some embodiments, the present disclosure teaches that the insert polynucleotide may also comprise copies of the target sequences for the introduced Cpf1 CRISPR systems, such that the insert polynucleotides are also processed in vivo to produce sticky ends. In some embodiments, the present disclosure teaches methods of targeting Cpf1 endonucleases such that they are position in an inwardly facing inverse orientation that ensures that digested insert polynucleotides are no longer substrates for the Cpf1 endonucleases.

In some embodiments, the present disclosure teaches that the specific targeting methods of the present disclosure for the digestion of the insert DNA and the genomic DNA, ensure that the resulting in vivo reactions proceed in a single direction (e.g., that ligated sticky ends are not subsequently re-digested by the Cpf1 endonuclease). In some embodiments, the present disclosure teaches that ensuring directionality in the digestion reactions improves the efficiency of the gene editing reactions.

Thus in some embodiments, the present disclosure teaches that the DNA inserts of the present disclosure also comprise two copies of the first target sequence positioned in an inwardly facing inverse orientation, such that cleavage of said insert polynucleotide by the Cpf1 endonuclease removes the first and second copies of the first target site from the insert polynucleotide.

In some embodiments, the in vivo CLIC methods of the present disclosure rely on endogenous DNA ligase activity to ligate to annealed sticky ends. In other embodiments, the present disclosure teaches introducing other ligase function into the edited cells. Thus, in some embodiments, the present disclosure teaches that the one or more vectors of the CLIC method comprise a fifth polynucleotide encoding a DNA ligase.

In some embodiments, the present disclosure teaches T4 and T7 ligases.

In some embodiments of the in vivo CLIC method, the present disclosure teaches that the Cpf1 endonuclease is non-naturally occurring. In other embodiments of the in vivo CLIC method, the present disclosure teaches that the Cpf1 endonuclease is naturally occurring and/or endogenous.

In some embodiments of the in vivo CLIC method, the present disclosure teaches that the crRNA is non-naturally occurring. In other embodiments of the in vivo CLIC method, the present disclosure teaches that the crRNA is naturally occurring and/or endogenous.

In some embodiments of the in vivo CLIC method, the present disclosure teaches that the ligase is non-naturally occurring. In other embodiments of the in vivo CLIC method, the present disclosure teaches that the ligase is naturally occurring and/or endogenous.

In some embodiments, the present disclosure teaches that the combination of the Cpf1 endonuclease, the crRNA, and (optionally) the ligase are non-naturally occurring.

In some embodiments, the present disclosure teaches a method for removing a transposon from the genome of a cell in vivo, said method comprising the steps of: a) introducing into the cell a Cpf1 CRISPR system comprising one or more vectors comprising: i) a first polynucleotide encoding a first crRNA that hybridizes to a first selected target sequence within the transposon; ii) a second polynucleotide encoding a second crRNA that hybridizes to a second selected target within the transposon; and ii) a third polynucleotide encoding a Cpf1 endonuclease; wherein components (a), (b), and (c) are expressed in the cell, and the Cpf1 endonuclease cleaves the cell's genome at the selected target sequences, thereby producing sticky ends on the cleaved ends of the cell's genome; wherein the first and second target sequences are positioned in an outwardly facing inverse orientation of within the transposon, such that removal of said transposon will also remove the first and second target sites from that portion of the genome; (b) annealing the resulting genome sticky ends to each other; and (c) ligating the annealed genome sticky ends from step (b); wherein the resulting ligated genome lacks said transposon.

### BRIEF DESCRIPTION OF THE FIGURES

FIG. 1A-B Comparison of the CRISPR Cas 9 and CRISPR Cpf1 systems of the present disclosure. A—Cas9 endonucleases are recruited to target dsDNA by tracrRNA and crRNA complexes. Cas 9 endonuclease produces blunt end cuts (dark arrows indicate cut locations). B-Cpf1 endonucleases only require crRNA guide polynucleotides. Cpf1 endonucleases produce sticky ends from staggered cuts depicted as dark arrows.

FIG. 2 Illustrates an embodiment of the present disclosure for CLIC single pot in vitro cloning using a Cpf1 endonuclease and ligase. A multiclonal site (MCS) or other non-desired insert is removed via Cpf1 digestion and is replaced with a gene of interest (GOI) insert. Cpf1 target sites located on DNA fragments slated for removal reduces nuclease interference with subsequent ligation reactions. Cpf1 endonuclease also reduces the incidence of MCS re-ligations.

FIG. 3 Illustrates another single pot in vitro cloning embodiment of the CLIC Cpf1 cloning methods of present disclosure. Various cassettes with different genes of interest (GOI) are flanked by Cpf1 target sites (top). After Cpf1-mediated cleavage of these cassettes (the source of these cassettes can be plasmids (as shown) or linear (e.g., PCR) fragments), the compatible ends facilitate ligation in the desired orientation and order (bottom). In this embodiment, Cpf1 target sites are located outside the GOI inserts, so as to not interfere with subsequent ligation steps. The resulting plasmid can be transformed into the host of interest (e.g., Escherichia coli).

FIG. 4A-C Illustrates several embodiments of the in vivo CLIC Cpf1 cloning methods of the present disclosure. A—Cpf1 can be designed to cut at two different target sites generating compatible ends. Using a ligase the double-strand break can be repaired by ligation, thereby removing the desired region (e.g., part of an open reading frame). Cpf1 target sites are located within the DNA region slated for removal in an outward facing orientation so as to reduce Cpf1 interference with subsequent ligation. B—Similarly, Cpf1 can be used to introduce new genetic material by cutting at two sites, generating a double stranded break (DSB) with two different sticky ends, and ligating a newly designed insert (e.g., an insert containing a beneficial SNP, such as the insert depicted in FIG. 4C). C—Using linear (PCR) fragments or an in vivo generated repair fragment with compatible overhangs (or also created using Cpf1 from a plasmid, as shown in FIG. 3) the DSB can be repaired by means of a ligase. Cpf1 enzymes are depicted in the target locations taught by some embodiments of the present disclosure (i.e., inside DNA regions being removed, and outside of inserts that will be ligated).

FIG. 5A-B Illustrates an embodiment of the CLIC two-part assembly methods of the present disclosure. A—Provides a high-level overview of the construct assembly. Black bent arrows represent Cpf1 cut sites. Shaded boxes represent distinct sticky end overhang sequences a′-c′. B—Provides additional sequence details for the cleavage portion indicated by the dotted boxes of FIG. 5A (OH=overhang). Note that while the overhangs are shown in different shades for clarity, the actual assembly is scarless since the overhangs are derived from the sequences themselves.

FIG. 6 Illustrates a method 100 for sequence-specific deletion of a target base DNA molecule, according to an embodiment of the present disclosure.

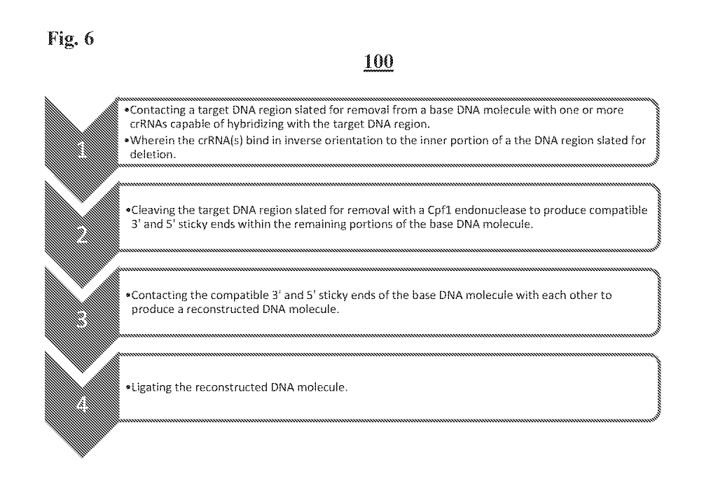

FIG. 7 Illustrates a method 200 for sequence-specific sequence replacement of a target base DNA molecule region slated for deletion with a new DNA insert molecule, according to an embodiment of the present disclosure.

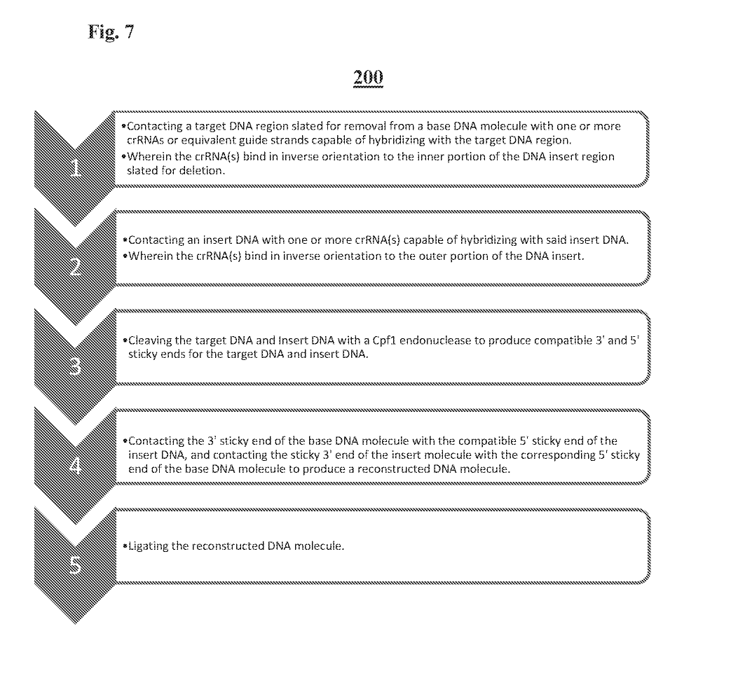

FIG. 8 Depicts the results of FnCpf1 purification. SDS page of BSA (Lane 1), and purified FnCpf1 according to SEQ ID No: 82 Arrow indicates expected size of Cpf1 polypeptide at 150 kDa.

FIG. 9 Depicts a quantification of purified FnCpf1 polypeptide using Bradford Assay. Purified FnCpf1 solution achieved concentration of 0.60 mg/ml.

FIG. 10 Depicts the results of in vitro CLIC Cpf1 digestion and re-ligation of PCR product. Agarose gel with Ethidium Bromide stain. Lane 1 shows expected 500 bp and 1500 bp digestion products from Cpf1 digestion. Lane 2 shows re-ligated 2000 bp product after Cpf1 inactivation and product ligation.

FIG. 11 Depicts the results of an in vitro CLIC reaction. Two PCR products were digested and ligated via compatible sticky ends with T7 DNA ligase in a single reaction. Lane 1 shows results of control reaction omitting T7 ligase. Lane 2 shows a band at 3000 bp, corresponding to ligated product.

FIG. 12 Depicts the results of an in vivo CLIC digestion of target resistance plasmids. Natively expressed Cpf1/crRNA complexes successfully targeted Wild Type resistance plasmids for reduced cell growth in antibiotic-containing media. Cpf1-mediated digestion could be abrogated by mutating the PAM of the resistance plasmid.

FIG. 13 Illustrates an embodiment of Cpf1 assembly methods of Example 8. Each panel provides an illustration of the experimental design described in Example 8. A chloramphenicol resistance gene was cloned into a kanamycin resistant backbone plasmid to create a dual resistance plasmid. Dual resistance plasmids were then transformed into bacteria, which was subsequently cultured in media augmented with kanamycin and chloramphenicol antibiotics. Resistant colonies indicated successful Cpf1 cloning assemblies.

FIG. 14 Depicts the results of the Cpf1 cloning assembly experiment of Example 8. The y-axis represents the number of recovered colonies growing in media augmented with kanamycin and chloramphenicol. Resistant colonies indicate successful Cpf1 cloning assemblies. The results showed a ligase-dependent assembly of dual resistance plasmids.

FIG. 15 Depicts the vector map for pJDI427. CRISPR landing sites used in the Cpf1 assembly are labeled as Guide A and Guide B.

FIG. 16 Depicts the vector map for pJDI429. CRISPR landing sites used in the Cpf1 assembly are labeled as Guide B and Guide C.

FIG. 17 Depicts the vector map for pJDI430. CRISPR landing sites used in the Cpf1 assembly are labeled as Guide D and Guide B.

FIG. 18 Depicts the vector map for pJDI431. CRISPR landing sites used in the Cpf1 assembly are labeled as Guide D and Guide C.

FIG. 19 Depicts the vector map for pJDI432. CRISPR landing sites used in the Cpf1 assembly are labeled as Guide A and Guide B.

FIG. 20 Depicts the vector map for pJDI434. CRISPR landing sites used in the Cpf1C assembly are labeled as Guide B and Guide C.

FIG. 21 Depicts the vector map for pJDI435. CRISPR landing sites used in the Cpf1 assembly are labeled as Guide D and Guide B.

FIG. 22 Depicts the vector map for pJDI436. CRISPR landing sites used in the Cpf1 assembly are labeled as Guide D and Guide C.

### DETAILED DESCRIPTION

### Definitions

While the following terms are believed to be well understood by one of ordinary skill in the art, the following definitions are set forth to facilitate explanation of the presently disclosed subject matter.

The term “a” or “an” refers to one or more of that entity, i.e., can refer to a plural referents. As such, the terms “a” or “an”, “one or more” and “at least one” are used interchangeably herein. In addition, reference to “an element” by the indefinite article “a” or “an” does not exclude the possibility that more than one of the elements is present, unless the context clearly requires that there is one and only one of the elements.

The term “prokaryotes” is art recognized and refers to cells, which contain no nucleus or other cell organelles. The prokaryotes are generally classified in one of two domains, the Bacteria and the Archaea. The definitive difference between organisms of the Archaea and Bacteria domains is based on fundamental differences in the nucleotide base sequence in the 16S ribosomal RNA.

A “eukaryote” is any organism whose cells contain a nucleus and other organelles enclosed within membranes. Eukaryotes belong to the taxon Eukarya or Eukaryota. The defining feature that sets eukaryotic cells apart from prokaryotic cells (the aforementioned Bacteria and Archaea) is that they have membrane-bound organelles, especially the nucleus, which contains the genetic material, and is enclosed by the nuclear envelope.

The term “Archaea” refers to a categorization of organisms of the division Mendosicutes, typically found in unusual environments and distinguished from the rest of the prokaryotes by several criteria, including the number of ribosomal proteins and the lack of muramic acid in cell walls. On the basis of ssrRNA analysis, the Archaea consist of two phylogenetically-distinct groups: Crenarchaeota and Euryarchaeota. On the basis of their physiology, the Archaea can be organized into three types: methanogens (prokaryotes that produce methane); extreme halophiles (prokaryotes that live at very high concentrations of salt (NaCl); and extreme (hyper) thermophilus (prokaryotes that live at very high temperatures). Besides the unifying archaeal features that distinguish them from Bacteria (i.e., no murein in cell wall, ester-linked membrane lipids, etc.), these prokaryotes exhibit unique structural or biochemical attributes which adapt them to their particular habitats. The Crenarchaeota consists mainly of hyperthermophilic sulfur-dependent prokaryotes and the Euryarchaeota contains the methanogens and extreme halophiles.

“Bacteria” or “eubacteria” refers to a domain of prokaryotic organisms. Bacteria include at least 11 distinct groups as follows: (1) Gram-positive (gram+) bacteria, of which there are two major subdivisions: (1) high G+C group (Actinomycetes, Mycobacteria, Micrococcus, others) (2) low G+C group (Bacillus, Clostridia, Lactobacillus, Staphylococci, Streptococci, Mycoplasmas); (2) Proteobacteria, e.g., Purple photosynthetic+non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia; (8) Green sulfur bacteria; (9) Green non-sulfur bacteria (also anaerobic phototrophs); (10) Radioresistant micrococci and relatives; (11) Thermotoga and Thermosipho thermophiles.

The terms “genetically modified host cell,” “recombinant host cell,” and “recombinant strain” are used interchangeably herein and refer to host cells that have been genetically modified by the cloning and transformation methods of the present disclosure. Thus, the terms include a host cell (e.g., bacteria, yeast cell, fungal cell, CHO, human cell, etc.) that has been genetically altered, modified, or engineered, such that it exhibits an altered, modified, or different genotype and/or phenotype (e.g., when the genetic modification affects coding nucleic acid sequences of the microorganism), as compared to the naturally-occurring microorganism from which it was derived. It is understood that the terms refer not only to the particular recombinant microorganism in question, but also to the progeny or potential progeny of such a microorganism.

The term “genetically engineered” may refer to any manipulation of a host cell's genome (e.g. by insertion or deletion of nucleic acids).

As used herein, the term “nucleic acid” refers to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA. It also includes modified nucleic acids such as methylated and/or capped nucleic acids, nucleic acids containing modified bases, backbone modifications, and the like. The terms “nucleic acid” and “nucleotide sequence” are used interchangeably.

As used herein, the term “gene” refers to any segment of DNA associated with a biological function. Thus, genes include, but are not limited to, coding sequences and/or the regulatory sequences required for their expression. Genes can also include non-expressed DNA segments that, for example, form recognition sequences for other proteins. Genes can be obtained from a variety of sources, including cloning from a source of interest or synthesizing from known or predicted sequence information, and may include sequences designed to have desired parameters.

As used herein, the term “homologous” or “homologue” or “ortholog” is known in the art and refers to related sequences that share a common ancestor or family member and are determined based on the degree of sequence identity. The terms “homology,” “homologous,” “substantially similar” and “corresponding substantially” are used interchangeably herein. They refer to nucleic acid fragments wherein changes in one or more nucleotide bases do not affect the ability of the nucleic acid fragment to mediate gene expression or produce a certain phenotype. These terms also refer to modifications of the nucleic acid fragments of the instant disclosure such as deletion or insertion of one or more nucleotides that do not substantially alter the functional properties of the resulting nucleic acid fragment relative to the initial, unmodified fragment. It is therefore understood, as those skilled in the art will appreciate, that the disclosure encompasses more than the specific exemplary sequences. These terms describe the relationship between a gene found in one species, subspecies, variety, cultivar or strain and the corresponding or equivalent gene in another species, subspecies, variety, cultivar or strain. For purposes of this disclosure, homologous sequences are compared. “Homologous sequences”, “homologues”, or “orthologs” are thought, believed, or known to be functionally related. A functional relationship may be indicated in any one of a number of ways, including, but not limited to: (a) degree of sequence identity and/or (b) the same or similar biological function. Preferably, both (a) and (b) are indicated. Homology can be determined using software programs readily available in the art, such as those discussed in Current Protocols in Molecular Biology (F. M. Ausubel et al., eds., 1987) Supplement 30, section 7.718, Table 7.71. Some alignment programs are MacVector (Oxford Molecular Ltd, Oxford, U.K.), ALIGN Plus (Scientific and Educational Software, Pennsylvania) and AlignX (Vector NTI, Invitrogen, Carlsbad, Calif.). Another alignment program is Sequencher (Gene Codes, Ann Arbor, Mich.), using default parameters.

As used herein, the term “nucleotide change” refers to, e.g., nucleotide substitution, deletion, and/or insertion, as is well understood in the art. For example, mutations contain alterations that produce silent substitutions, additions, or deletions, but do not alter the properties or activities of the encoded protein or how the proteins are made.

As used herein, the term “protein modification” refers to, e.g., amino acid substitution, amino acid modification, deletion, and/or insertion, as is well understood in the art.

As used herein, the term “at least a portion” or “fragment” of a nucleic acid or polypeptide means a portion having the minimal size characteristics of such sequences, or any larger fragment of the full length molecule, up to and including the full length molecule. A fragment of a polynucleotide of the disclosure may encode a biologically active portion of a genetic regulatory element. A biologically active portion of a genetic regulatory element can be prepared by isolating a portion of one of the polynucleotides of the disclosure that comprises the genetic regulatory element and assessing activity as described herein. Similarly, a portion of a polypeptide may be 4 amino acids, 5 amino acids, 6 amino acids, 7 amino acids, and so on, going up to the full length polypeptide. The length of the portion to be used will depend on the particular application. A portion of a nucleic acid useful as a hybridization probe may be as short as 12 nucleotides; in some embodiments, it is 20 nucleotides. A portion of a polypeptide useful as an epitope may be as short as 4 amino acids. A portion of a polypeptide that performs the function of the full-length polypeptide would generally be longer than 4 amino acids.

For PCR amplifications of the polynucleotides disclosed herein, oligonucleotide primers can be designed for use in PCR reactions to amplify corresponding DNA sequences from cDNA or genomic DNA extracted from any organism of interest. Methods for designing PCR primers and PCR cloning are generally known in the art and are disclosed in Sambrook et al. (2001) Molecular Cloning: A Laboratory Manual (3rd ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

The term “primer” as used herein refers to an oligonucleotide which is capable of annealing to the amplification target allowing a DNA polymerase to attach, thereby serving as a point of initiation of DNA synthesis when placed under conditions in which synthesis of primer extension product is induced, i.e., in the presence of nucleotides and an agent for polymerization such as DNA polymerase and at a suitable temperature and pH. The (amplification) primer is preferably single stranded for maximum efficiency in amplification. Preferably, the primer is an oligodeoxyribonucleotide. The primer must be sufficiently long to prime the synthesis of extension products in the presence of the agent for polymerization. The exact lengths of the primers will depend on many factors, including temperature and composition (A/T vs. G/C content) of primer. A pair of bi-directional primers consists of one forward and one reverse primer as commonly used in the art of DNA amplification such as in PCR amplification.

The terms “stringency” or “stringent hybridization conditions” refer to hybridization conditions that affect the stability of hybrids, e.g., temperature, salt concentration, pH, formamide concentration and the like. These conditions are empirically optimized to maximize specific binding and minimize non-specific binding of primer or probe to its target nucleic acid sequence. The terms as used include reference to conditions under which a probe or primer will hybridize to its target sequence, to a detectably greater degree than other sequences (e.g. at least 2-fold over background). Stringent conditions are sequence dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH. The Tm is the temperature (under defined ionic strength and pH) at which 50% of a complementary target sequence hybridizes to a perfectly matched probe or primer. Typically, stringent conditions will be those in which the salt concentration is less than about 1.0 M Na+ ion, typically about 0.01 to 1.0 M Na+ ion concentration (or other salts) at pH 7.0 to 8.3 and the temperature is at least about 30° C. for short probes or primers (e.g. 10 to 50 nucleotides) and at least about 60° C. for long probes or primers (e.g. greater than 50 nucleotides). Stringent conditions may also be achieved with the addition of destabilizing agents such as formamide. Exemplary low stringent conditions or “conditions of reduced stringency” include hybridization with a buffer solution of 30% formamide, 1 M NaCl, 1% SDS at 37° C. and a wash in 2×SSC at 40° C. Exemplary high stringency conditions include hybridization in 50% formamide, IM NaCl, 1% SDS at 37° C., and a wash in 0.1×SSC at 60° C. Hybridization procedures are well known in the art and are described by e.g. Ausubel et al., 1998 and Sambrook et al., 2001. In some embodiments, stringent conditions are hybridization in 0.25 M Na2HPO4 buffer (pH 7.2) containing 1 mM Na2EDTA, 0.5-20% sodium dodecyl sulfate at 45° C., such as 0.5%, 1%, 2%, 3%, 4%, 5%, 6%, 7%, 8%, 9%, 10%, 11%, 12%, 13%, 14%, 15%, 16%, 17%, 18%, 19% or 20%, followed by a wash in 5×SSC, containing 0.1% (w/v) sodium dodecyl sulfate, at 55° C. to 65° C.

As used herein, “promoter” refers to a DNA sequence capable of controlling the expression of a coding sequence or functional RNA. The promoter sequence may consist of proximal and more distal upstream elements, the latter elements often referred to as enhancers. Accordingly, an “enhancer” is a DNA sequence that can stimulate promoter activity, and may be an innate element of the promoter or a heterologous element inserted to enhance the level or tissue specificity of a promoter.

As used herein, the term “heterologous” refers to a nucleic acid sequence, which is not naturally found in the particular organism.

As used herein, the term “endogenous,” “endogenous gene,” refers to the naturally occurring copy of a gene.

As used herein, the term “naturally occurring” refers to a gene derived from a naturally occurring source. In some embodiments, a naturally occurring gene refers to a gene of a wild type (non-transgene) gene, whether located in its endogenous setting within the source organism, or if placed in a “heterologous” setting, when introduced in a different organism. Thus, for the purposes of this disclosure, a “non-naturally occurring” gene is a gene that has been synthesized, mutated, or otherwise modified to have a different sequence from known natural genes. In some embodiments, the modification may be at the protein level (e.g., amino acid substitutions). In other embodiments, the modification may be at the DNA level, without any effect on protein sequence (e.g., codon optimization). In some embodiments, the non-naturally occurring gene may be a chimeric gene as described infra.

As used herein, the term “exogenous” is used interchangeably with the term “heterologous,” and refers to a substance coming from some source other than its native source. For example, the terms “exogenous protein,” or “exogenous gene” refer to a protein or gene from a non-native source or location, and that have been artificially supplied to a biological system. Artificially mutated variants of endogenous genes are considered “exogenous” for the purposes of this disclosure.

As used herein, the phrases “recombinant construct”, “expression construct”, “chimeric construct”, “construct”, and “recombinant DNA construct” are used interchangeably herein. A recombinant construct comprises an artificial combination of nucleic acid fragments, e.g., regulatory and coding sequences that are not found together in nature. For example, a chimeric construct may comprise regulatory sequences and coding sequences that are derived from different sources, or regulatory sequences and coding sequences derived from the same source, but arranged in a manner different than that found in nature. Such construct may be used by itself or may be used in conjunction with a vector. If a vector is used then the choice of vector is dependent upon the method that will be used to transform host cells as is well known to those skilled in the art. For example, a plasmid vector can be used. The skilled artisan is well aware of the genetic elements that must be present on the vector in order to successfully transform, select and propagate host cells comprising any of the isolated nucleic acid fragments of the disclosure. The skilled artisan will also recognize that different independent transformation events will result in different levels and patterns of expression (Jones et al., (1985) EMBO J. 4:2411-2418; De Almeida et al., (1989) Mol. Gen. Genetics 218:78-86), and thus that multiple events must be screened in order to obtain lines displaying the desired expression level and pattern. Such screening may be accomplished by Southern analysis of DNA, Northern analysis of mRNA expression, immunoblotting analysis of protein expression, or phenotypic analysis, among others. Vectors can be plasmids, viruses, bacteriophages, pro-viruses, phagemids, transposons, artificial chromosomes, and the like, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that is not autonomously replicating. As used herein, the term “expression” refers to the production of a functional end-product e.g., an mRNA or a protein (precursor or mature).

The term “operably linked” means in this context the sequential arrangement of the promoter polynucleotide according to the disclosure with a further oligo- or polynucleotide, resulting in transcription of said further polynucleotide. In some embodiments, the promoter sequences of the present disclosure are inserted just prior to a gene's 5′UTR, or open reading frame. In other embodiments, the operably linked promoter sequences and gene sequences of the present disclosure are separated by one or more linker nucleotides.

The term “CRISPR RNA” or “crRNA” refers to the guide RNA strand responsible for hybridizing with target DNA sequences, and recruiting CRISPR endonucleases. crRNAs may be naturally occurring, or may be synthesized according to any known method of producing RNA. In some embodiments, the term crRNA, guide RNA and sgRNA are equivalent for Cpf1, and may be interchangeably used throughout this document.

The term “guide sequence” or “spacer” refers to the portion of a crRNA that is responsible for hybridizing with the target DNA.

The term “protospacer” refers to the DNA sequence targeted by a crRNA guide strand. In some embodiments, the protospacer sequence hybridizes with the crRNA guide sequence/spacer of a CRISPR complex.

The term “seed region” refers to the ribonucleic sequence responsible for initial complexation between a DNA sequence and a CRISPR ribonucleoprotein complex. Mismatches between the seed region and a target DNA sequence have a stronger effect on target site recognition and cleavage than the remainder of the crRNA/sgRNA sequence. In some embodiments, a single mismatch in the seed region of a crRNA can render a CRISPR complex inactive at that binding site. In some embodiments, the seed regions for Cas9 endonucleases are located along that last 12 nts of the 3′ portion of the guide sequence, which correspond (hybridize) to the portion of the protospacer target sequence that is adjacent to the PAM. In some embodiments, the seed regions for Cpf1 endonucleases are located along the first 5 nts of the 5′ portion of the guide strand, which correspond (hybridize) to the portion of the protospacer target sequence adjacent to the PAM.

The term “Guide RNA” or “gRNA” as used herein refers to an RNA sequence or combination of sequences capable of recruiting a CRISPR endonuclease to a target sequence. Thus as used herein, a guide RNA can be a natural or synthetic crRNA (e.g., for Cpf1), a natural or synthetic crRNA/tracrRNA hybrid (e.g., for Cas9), or a single-guide RNA (sgRNA).

The term “CRISPR complex” as used herein, refers to a CRISPR endonuclease that is operably associated with a Guide RNA. In some embodiments, a CRISPR complex of the present disclosure is a Cpf1 endonuclease operable associated with a crRNA, such that the complex is capable of cleaving a DNA region targeted by the crRNA. In some embodiments the terms CRISPR complex and CRISPR system are used interchangeably.

The term “CRISPR landing site” as used herein, refers to a DNA sequence capable of being targeted by a CRISPR complex. Thus, in some embodiments, a CRISPR landing site comprises a proximately placed protospacer/Protopacer Adjacent Motif combination sequence that is capable of being cleaved a CRISPR endonuclease complex. The term “validated CRISPR landing site” refers to a CRISPR landing site for which there exists a guide RNA capable of inducing high efficiency cleaving of said sequence. Thus, the term validated should be interpreted as meaning that the sequence has been previously shown to be cleavable by a CRISPR complex. Each “validated CRISPR landing site” will by definition confirm the existence of a tested guide RNA associated with the validation.

The term “sticky end(s)” refers to double stranded polynucleotide molecule end that comprises a sequence overhang. In some embodiments, the sticky end can be a dsDNA molecule end with a 5′ or 3′ sequence overhang. In some embodiments, the sticky ends of the present disclosure are capable of hybridizing with compatible sticky ends of the same or other molecules. Thus in one embodiment, a sticky end on the 3′ of a first DNA fragment may hybridize with a compatible sticky end on a second DNA fragment. In some embodiments, these hybridized sticky ends can be sewn together by a ligase. In other embodiments, the sticky ends might require extension of the overhangs to complete the dsDNA molecule prior to ligation. The term “genetic scar(s)” refers to any undesirable sequence introduced into a nucleic acid sequence by DNA manipulation methods. For example, in some embodiments, the present disclosure teaches genetic scars such as restriction enzyme binding sites, sequence adapters or spacers to accommodate cloning, TA-sites, scars left over from NHEJ, etc. In some embodiments, the present disclosure teaches methods of scarless cloning and gene editing.

As used herein the term “targeted” refers to the expectation that one item or molecule will interact with another item or molecule with a degree of specificity, so as to exclude non-targeted items or molecules. For example, a first polynucleotide that is targeted to a second polynucleotide, according to the present disclosure has been designed to hybridize with the second polynucleotide in a sequence specific manner (e.g., via Watson-crick base pairing). In some embodiments, the selected region of hybridization is designed so as to render the hybridization unique to the one, or more targeted regions. A second polynucleotide can cease to be a target of a first targeting polynucleotide, if its targeting sequence (region of hybridization) is mutated, or is otherwise removed/separated from the second polynucleotide.

### Gene Editing

The principles of in vivo CRISPR-based editing largely rely on natural cellular DNA repair systems. Double-stranded dsDNA breaks introduced by nucleases are repaired by either non-homologous end-joining (NHEJ) or homology-directed repair (HDR), or single strand annealing (SSA), or microhomology end joining (MMEJ).

HDR relies on a template DNA containing sequences homologous to the region surrounding the targeted site of DNA cleavage. Cellular repair proteins use the homology between the exogenously supplied or endogenous DNA sequences and the site surrounding the DNA break to repair the dsDNA break, replacing the break with the sequence on the template DNA. Failure to integrate the template DNA however, can result in NHEJ, MMEJ, or SSA. NHEJ, MMEJ and SSA are error-prone processes that are often accompanied by insertion or deletion of nucleotides (indels) at the target site, resulting in genetic knockout (silencing) of the targeted region of the genome due to frameshift mutations or insertions of a premature stop codon. Cpf1-mediated editing can also function via traditional hybridization of overhangs created by the endonuclease, followed by ligation.

CRISPR endonucleases are also useful for in vitro DNA manipulations, as discussed in later sections of this disclosure.

DNA Nucleases

In some embodiments, the present disclosure teaches methods and compositions for gene editing utilizing DNA nucleases. In some embodiments, the present disclosure teaches methods of gene editing using any targetable DNA nuclease (e.g., Cpf1, Cas9, or other natural or synthetic Targetable Enzyme).

CRISPR systems, transcription activator-like effector nucleases (TALENs), zinc finger nucleases (ZFNs), and FokI restriction enzymes are some of the sequence-specific nucleases that have been used as gene editing tools. These enzymes are able to target their nuclease activities to desired target loci through interactions with guide regions engineered to recognize sequences of interest. In some embodiments, the present disclosure teaches CRISPR-based gene editing methods

CRISPR Systems

CRISPR (Clustered Regularly Interspaced Short Palindromic Repeats) and CRISPR-associated (cas) endonucleases were originally discovered as adaptive immunity systems evolved by bacteria and archaea to protect against viral and plasmid invasion. Naturally occurring CRISPR/Cas systems in bacteria are composed of one or more Cas genes and one or more CRISPR arrays consisting of short palindromic repeats of base sequences separated by genome-targeting sequences acquired from previously encountered viruses and plasmids (called spacers). (Wiedenheft, B., et. al. Nature. 2012; 482:331; Bhaya, D., et. al., Annu. Rev. Genet. 2011; 45:231; and Terms, M. P. et. al., Curr. Opin. Microbiol. 2011; 14:321). Bacteria and archaea possessing one or more CRISPR loci, respond to viral or plasmid challenge by integrating short fragments of foreign sequence (protospacers) into the host chromosome at the proximal end of the CRISPR array. Transcription of CRISPR loci generates a library of CRISPR-derived RNAs (crRNAs) containing sequences complementary to previously encountered invading nucleic acids (Haurwitz, R. E., et. al., Science. 2012:329; 1355; Gesner, E. M., et. al., Nat. Struct. Mol. Biol. 2001:18; 688; Jinek, M., et. al., Science. 2012:337; 816-21). Target recognition by crRNAs occurs through complementary base pairing with target DNA, which directs cleavage of foreign sequences by means of Cas proteins. (Jinek et. al. 2012 “A Programmable dual-RNA-guided DNA endonuclease in adaptive bacterial immunity.” Science. 2012:337; 816-821).

There are at least five main CRISPR system types (Type I, II, III, IV and V) and at least 16 distinct subtypes (Makarova, K. S., et al., Nat Rev Microbiol. 2015. Nat. Rev. Microbiol. 13, 722-736). CRISPR systems are also classified based on their effector proteins. Class 1 systems possess multi-subunit crRNA-effector complexes, whereas in class 2 systems all functions of the effector complex are carried out by a single protein (e.g., Cas9 or Cpf1). In some embodiments, the present disclosure teaches using type II and/or type V single-subunit effector systems. Thus, in some embodiments, the present disclosure teaches using class 2 CRISPR systems.

CRISPR Cas9

In some embodiments, the present disclosure teaches methods of gene editing using a Type II CRISPR system. In some embodiments, the present disclosure teaches Cas9 Type II CRISPR systems. Type II systems rely on a 1) single endonuclease protein, ii) a transactiving crRNA (tracrRNA), and iii) a crRNA where a ˜20-nucleotide (nt) portion of the 5′ end of crRNA is complementary to a target nucleic acid. The region of a CRISPR crRNA strand that is complementary to its target DNA protospacer is hereby referred to as “guide sequence.”

In some embodiments, the tracrRNA and crRNA components of a Type II system can be replaced by a single-guide RNA (sgRNA). The sgRNA can include, for example, a nucleotide sequence that comprises an at least 12-20 nucleotide sequence complementary to the target DNA sequence (guide sequence) and can include a common scaffold RNA sequence at its 3′ end. As used herein, “a common scaffold RNA” refers to any RNA sequence that mimics the tracrRNA sequence or any RNA sequences that function as a tracrRNA.

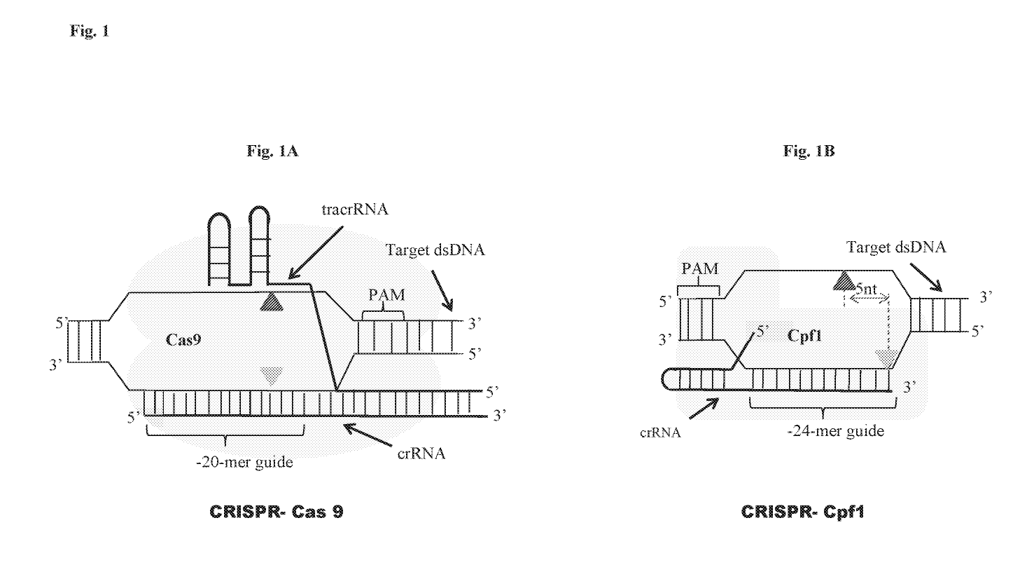

Cas9 endonucleases produce blunt end DNA breaks, and are recruited to target DNA by a combination of a crRNA and a tracrRNA oligos, which tether the endonuclease via complementary hybridization of the RNA CRISPR complex. (see solid triangle arrows in FIG. 1A)

In some embodiments, DNA recognition by the crRNA/endonuclease complex requires additional complementary base-pairing with a protospacer adjacent motif (PAM) (e.g., 5′-NGG-3′) located in a 3′ portion of the target DNA, downstream from the target protospacer. (Jinek, M., et. al., Science. 2012:337; 816-821). In some embodiments, the PAM motif recognized by a Cas9 varies for different Cas9 proteins.

In some embodiments, one skilled in the art can appreciate that the Cas9 disclosed herein can be any variant derived or isolated from any source. For example, in some embodiments, the Cas9 peptide of the present disclosure can include one or more of SEQ ID Nos selected from SEQ ID NO: 1, SEQ ID NO: 2, SEQ ID NO: 3, SEQ ID NO: 4, SEQ ID NO: 5, and SEQ ID NO: 6. In other embodiments, the Cas9 peptide of the present disclosure can include one or more of the mutations described in the literature, including but not limited to the functional mutations described in: Fonfara et al. Nucleic Acids Res. 2014 February; 42(4):2577-90; Nishimasu H. et al. Cell. 2014 Feb. 27; 156(5):935-49; Jinek M. et al. Science. 2012 337:816-21; and Jinek M. et al. Science. 2014 Mar. 14; 343(6176); see also U.S. patent application Ser. No. 13/842,859 filed Mar. 15, 2013, which is hereby incorporated by reference; further, see U.S. Pat. Nos. 8,697,359; 8,771,945; 8,795,965; 8,865,406; 8,871,445; 8,889,356; 8,895,308; 8,906,616; 8,932,814; 8,945,839; 8,993,233; and 8,999,641, which are all hereby incorporated by reference. Thus, in some embodiments, the systems and methods disclosed herein can be used with the wild type Cas9 protein having double-stranded nuclease activity, Cas9 mutants that act as single stranded nickases, or other mutants with modified nuclease activity.

In some embodiments, the present disclosure teaches methods of in vivo and in vitro genetic manipulation using modified Cas9 endonucleases to produce a Targetable Enzyme. For example, in some embodiments, the present disclosure teaches use of Cas9 nickases. In some embodiments, the present disclosure teaches Cas9 chimeric fusion proteins with nuclease domains that produce sticky domains. That is, in some embodiments, the present disclosure teaches enzymatically inactive Cas9 domains translationally fused (e.g., N- or C-terminal fusions) with a DNA nuclease capable of producing 3′ or 5′ overhangs. The present disclosure teaches methods of creating chimeric proteins in later sections of the document.

### CRISPR Cpf1

In other embodiments, the present disclosure teaches methods of gene editing using a Type V CRISPR system. In some embodiments, the present disclosure teaches methods of using CRISPR from Prevotella and Francisella 1 (Cpf1).

The Cpf1 CRISPR systems of the present disclosure comprise 1) a single endonuclease protein, and ii) a crRNA, wherein a portion of the 3′ end of crRNA contains the guide sequence complementary to a target nucleic acid. In this system, the Cpf1 nuclease is directly recruited to the target DNA by the crRNA (see solid triangle arrows in FIG. 1B). In some embodiments, guide sequences for Cpf1 must be at least 12nt, 13nt, 14nt, 15nt, or 16nt in order to achieve detectable DNA cleavage, and a minimum of 14nt, 15nt, 16nt, 17nt, or 18nt to achieve efficient DNA cleavage.

The Cpf1 systems of the present disclosure differ from Cas9 in a variety of ways. First, unlike Cas9, Cpf1 does not require a separate tracrRNA for cleavage. In some embodiments, Cpf1 crRNAs can be as short as about 42-44 bases long—of which 23-25 nts are guide sequence and 19 nts are the constitutive direct repeat sequence. In contrast, the combined Cas9 tracrRNA and crRNA synthetic sequences can be about 100 bases long. In some embodiments, the present disclosure will refer to a crRNA for Cpf1 as a “guide RNA.”

Second, Cpf1 has different PAM requirements. For example, FnCpf1 prefers a “TTN” PAM motif that is located 5′ upstream of its target. This is in contrast to the “NGG” PAM motifs located on the 3′ of the target DNA for Cas9 systems. In some embodiments, the uracil base immediately preceding the guide sequence cannot be substituted (Zetsche, B. et al. 2015. “Cpf1 Is a Single RNA-Guided Endonuclease of a Class 2 CRISPR-Cas System” Cell 163, 759-771, which is hereby incorporated by reference in its entirety for all purposes).

Third, the cut sites for Cpf1 are staggered by about 3-5 bases, which create “sticky ends” (Kim et al., 2016. “Genome-wide analysis reveals specificities of Cpf1 endonucleases in human cells” published online Jun. 6, 2016). These sticky ends with 3-5 bp overhangs are thought to facilitate NHEJ-mediated-ligation, and improve gene editing of DNA fragments with matching ends. The cut sites are in the 3′ end of the target DNA, distal to the 5′ end where the PAM is. The cut positions usually follow the 18th base on the non-hybridized strand and the corresponding 23rd base on the complementary strand hybridized to the crRNA (FIG. 1B).

Fourth, in Cpf1 complexes, the “seed” region is located within the first 5 nt of the guide sequence. Cpf1 crRNA seed regions are highly sensitive to mutations, and even single base substitutions in this region can drastically reduce cleavage activity (see Zetsche B. et al. 2015 “Cpf1 Is a Single RNA-Guided Endonuclease of a Class 2 CRISPR-Cas System” Cell 163, 759-771). Critically, unlike the Cas9 CRISPR target, the cleavage sites and the seed region of Cpf1 systems do not overlap. Additional guidance on designing Cpf1 crRNA targeting oligos is available on (Zetsche B. et al. 2015. “Cpf1 Is a Single RNA-Guided Endonuclease of a Class 2 CRISPR-Cas System” Cell 163, 759-771).

Persons skilled in the art will appreciate that the Cpf1 disclosed herein can be any variant derived or isolated from any source. For example, in some embodiments, the Cpf1 peptide of the present disclosure can include one or more of SEQ ID Nos selected from SEQ ID NO: 7, 8, 9, 10, 11, 12, 13, 14, 15, 16, 17, 18, 19, 20, 21, 22, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78 or 82, or any variants thereof. In some embodiments, the Cpf1 nuclease of the present disclosure comprises the sequence in SEQ ID NO: 7. In some embodiments, the Cpf1 nuclease of the present disclosure comprises the sequence in SEQ ID NO: 82.

### Modified Non-Naturally Occurring CRISPR Variants

In some embodiments, the present disclosure teaches modified CRISPR Cpf1 variants for improved gene editing efficiency. As used herein, the term “Cpf1” should be broadly construed to include both naturally occurring Cpf1 polypeptides, as well as mutated/chimeric variants thereof. In some embodiments, the present disclosure teaches methods of cleaving target DNA via targeted Cpf1 complexes, and then ligating the resulting sticky ends with DNA inserts. In some embodiments, the present disclosure teaches methods of providing a Cpf1 complex to cleave the target DNA, and a ligase to “sew” the DNA back together. In other embodiments, the present disclosure teaches modified Cpf1 complexes that include a tethered ligase enzyme.

Ligases

As used herein, the term “ligase” can comprise any number of enzymatic or non-enzymatic reagents. For example, ligase is an enzymatic ligation reagent or catalyst that, under appropriate conditions, forms phosphodiester bonds between the 3′-OH and the 5′-phosphate of adjacent nucleotides in DNA molecules, RNA molecules, or hybrids.

In some embodiments, the present disclosure teaches the use of enzymatic ligases. Compatible temperature sensitive enzymatic ligases, include, but are not limited to, bacteriophage T4 ligase, T7 ligase, and E. coli ligase. Thermostable ligases include, but are not limited to, Afu ligase, Taq ligase, Tfl ligase, Tth ligase, Tth HB8 ligase, Thermus species AK16D ligase and Pfu ligase (see for example Published P.C.T. Application WO/2000/026381, Wu et al., Gene, 76(2):245-254, (1989), and Luo et al., Nucleic Acids Research, 24(15): 3071-3078 (1996)). The skilled artisan will appreciate that any number of thermostable ligases can be obtained from thermophilic or hyperthermophilic organisms, for example, certain species of eubacteria and archaea; and that such ligases can be employed in the disclosed methods and kits. In some embodiments, reversibly inactivated enzymes (see for example U.S. Pat. No. 5,773,258) can be employed in some embodiments of the present teachings.

In other embodiments, the present disclosure teaches the use of chemical ligation agents. Chemical ligation agents include, without limitation, activating, condensing, and reducing agents, such as carbodiimide, cyanogen bromide (BrCN), N-cyanoimidazole, imidazole, 1-methylimidazole/carbodiimide/cystamine, dithiothreitol (DTT) and ultraviolet light. Autoligation, i.e., spontaneous ligation in the absence of a ligating agent, is also within the scope of the teachings herein. Detailed protocols for chemical ligation methods and descriptions of appropriate reactive groups can be found in, among other places, Xu et al., Nucleic Acid Res., 27:875-81 (1999); Gryaznov and Letsinger, Nucleic Acid Res. 21:1403-08 (1993); Gryaznov et al., Nucleic Acid Res. 22:2366-69 (1994); Kanaya and Yanagawa, Biochemistry 25:7423-30 (1986); Luebke and Dervan, Nucleic Acids Res. 20:3005-09 (1992); Sievers and von Kiedrowski, Nature 369:221-24 (1994); Liu and Taylor, Nucleic Acids Res. 26:3300-04 (1999); Wang and Kool, Nucleic Acids Res. 22:2326-33 (1994); Purmal et al., Nucleic Acids Res. 20:3713-19 (1992); Ashley and Kushlan, Biochemistry 30:2927-33 (1991); Chu and Orgel, Nucleic Acids Res. 16:3671-91 (1988); Sokolova et al., FEBS Letters 232:153-55 (1988); Naylor and Gilham, Biochemistry 5:2722-28 (1966); and U.S. Pat. No. 5,476,930.

In some embodiments, the methods, kits and compositions of the present disclosure are also compatible with photoligation reactions. Photoligation using light of an appropriate wavelength as a ligation agent is also within the scope of the teachings. In some embodiments, photoligation comprises probes comprising nucleotide analogs, including but not limited to, 4-thiothymidine, 5-vinyluracil and its derivatives, or combinations thereof. In some embodiments, the ligation agent comprises: (a) light in the UV-A range (about 320 nm to about 400 nm), the UV-B range (about 290 nm to about 320 nm), or combinations thereof, (b) light with a wavelength between about 300 nm and about 375 nm, (c) light with a wavelength of about 360 nm to about 370 nm; (d) light with a wavelength of about 364 nm to about 368 nm, or (e) light with a wavelength of about 366 nm. In some embodiments, photoligation is reversible. Descriptions of photoligation can be found in, among other places, Fujimoto et al., Nucl. Acid Symp. Ser. 42:39-40 (1999); Fujimoto et al., Nucl. Acid Res. Suppl. 1:185-86 (2001); Fujimoto et al., Nucl. Acid Suppl., 2:155-56 (2002); Liu and Taylor, Nucl. Acid Res. 26:3300-04 (1998) and on the world wide web at: sbchem.kyoto-u.ac.jp/saito-lab.

Chimeric CRISPR Polypetides (e.g. Cpf1-Ligase Polypeptides).

In some embodiments, the present disclosure teaches fusing a Cpf1 or other CRISPR polypeptide with a polypeptide with ligase activity. In some embodiments, ligases fused to Cpf1 complexes are enzymatic ligases. Methods for creating chimeric fusions are well-known in the art, and are discussed in Sambrook et al. (2001) Molecular Cloning: A Laboratory Manual (3rd ed., Cold Spring Harbor Laboratory Press, Plainview, N.Y.).

In some embodiments, a linker is used to genetically fuse an enzymatic ligase to a Cpf1 or other Targetable Enzyme gene to create an engineered, non-naturally occurring protein. In some embodiments, units are linked using a chemical compound. In some embodiments, the linker is an inorganic compound. In some embodiments, the linker is an organic compound. In some embodiments, the linker is a hybrid organic and inorganic compound.

In some embodiments, the linker is covalently bonded to Cpf1 or other Targetable Enzyme and the ligase. In some embodiments, the genes are genetically fused. In some embodiments, the linker is translationally fused to Cpf1 or other Targetable Enzyme and the ligase. In some embodiments, linkage occurs from about the 3′ end of Cpf1 sequence to about the 5′ end of the ligase sequence. In some embodiments, linkage occurs from about the 3′ end of the ligase sequence to about the 5′ prime end of Cpf1 or other Targetable Enzyme. In some embodiments, the linker is included within the open reading frame. In some embodiments, linkage occurs at any suitable position on Cpf1 or other Targetable Enzyme.

In some embodiments, the linker is an amino acid sequence. In some embodiments, the amino acids of the linker can include one or more amino acids selected from the group consisting of: glycine, alanine, serine, threonine, cysteine, valine, leucine, isoleucine, methionine, proline, phenylalanine, tyrosine, tryptophan, aspartic acid, glutamic acid, asparagine, glutamine, histidine, lysine, arginine, and/or combinations thereof. In some embodiments, the linker amino acid sequence is fused to Cpf1 or other Targetable Enzyme and the ligase.

As discussed in earlier sections, some embodiments of the present disclosure teach methods of creating other Cpf1 or Cas9 chimeric fusion proteins. That is, in some embodiments, the present disclosure teaches Cpf1 and/or Cas9 proteins translationally fused to one or more DNA nuclease domains capable of producing DNA cuts with 3′ or 5′ overhangs. In some embodiments, these synthetically produced CRISPR fusions with DNA nucleases are referred to as Targetable Enzymes.

Fusion of protein subunits of a complex has been performed on other systems and can be accomplished with the constructs disclosed herein by one skilled in the art with knowledge of the nucleic acid sequences to be fused to the Cas9 or Cpf1. Examples of genetic fusion of proteins using an amino acid sequence include the following, which are herein incorporated by reference in their entirety: (1) Martin, A. et al. Nature 2005 Oct. 20; 437:1115-1120); (2) Wang, F. et al. Nature 2014 Aug. 28; 512:441-444; (3) Schmitz, K. R. and Sauer, R. T. Molecular Microbiology. 2014 Jul. 13; 93(4):617-628; (4) Wang, Q. et al. Chem. Commun. 2014 Mar. 3; 50:4299-4301; (5) Andre, C. et al. S. PNAS. 2013 Feb. 19, 110(8):3191-3196; (7) Weidle, U. H. et al. Cancer Genomics and Proteomics. 2012 9(6):357-372).

Examples of fusing an exogenous active domain to a separate protein to create a construct with activities of both units include the following, which is herein incorporated by reference: Wa, F. US. Pat. Pub. No. 20140273226. 2014 Sep. 18.

In some embodiments, the linker includes about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, or 300 amino acids, and all ranges and subranges there between.

Nuclear Localization Signal (NLS)

In some embodiments, viable genome-editing tools must be delivered to the nucleus of eukaryotic cells. In other embodiments, the complexes of the present disclosure must be delivered to organelles with genetic information (e.g., chloroplasts and/or mitochondria). In yet other embodiments, the genome-editing tools of the present disclosure are used in organisms without nuclei. Thus, in some embodiments, the present disclosure teaches chimeric Cpf1 polypeptides comprising one or more nuclear localization signals. A nuclear localization signal or sequence (NLS) is an amino acid sequence that ‘tags’ a protein for import into the cell nucleus by nuclear transport. In some embodiments, this signal consists of one or more short sequences of positively charged lysines or arginines exposed on the protein surface. Clusters of arginines or lysines in nucleus-targeted proteins signal the anchoring of these proteins to specialized transporter molecules found on the complex or in the cytoplasm. In some embodiments, one or more NLS can be genetically linked to one or more of the polypeptides disclosed herein. In some embodiments, the NLS is genetically linked to a Cpf1 protein. In some embodiments, the NLS is included within the open reading frame of the Cpf1 gene. In some embodiments, the NLS is genetically linked to the C-terminus and/or the N-terminus of a Cpf1 protein. In some embodiments, the NLS is included in the linker sequence connecting a Cpf1 protein to a fused protein or portion thereof (e.g., linker between Cpf1 and ligase).

The NLS can be, for example, one or more short sequences of positively charged lysines or arginines exposed on the protein surface; can be either monopartite or bipartite; can be either classical or nonclassical NLSs. Suitable NLSs can be, for example, a PY-NLS motif; PKKKRKV (SEQ ID NO:23); the acidic M9 domain of hnRNP A1, the sequence KIPIK (SEQ ID NO:24) of the yeast transcription repressor Matα2, the complex signals of U snRNPs, the RKRRR (SEQ ID NO:25) motif from Notch 1 protein, the KRKRK (SEQ ID NO:26) from Notch 2 protein, the RRKR (SEQ ID NO:27) motif from Notch3 protein, the RRRRR (SEQ ID NO: 28) motif from Notch4 protein, and any other NLSs from any nuclear proteins known or later discovered by those skilled in the art.

### CRISPR and Ligase Cloning and Gene Editing

In some embodiments, the present disclosure teaches a CRISPR and Ligase Cloning method (termed “CLIC”). CLIC is a method for DNA assembly that relies on the CRISPR nuclease Cpf1 to digest DNA molecules, leaving behind three-five base-pair sticky ends whose sequence can be selected by the user. These sticky ends are then ligated together with a DNA ligase in order to join two or more digested fragments into a fully assembled construct or genome. Due to the long (˜18 bp) and programmable recognition sequences of Cpf1, CLIC eliminates the requirement to remove restriction enzyme recognition sites from the DNA molecules being assembled. In some embodiments, CLIC can be performed either in vitro for the scarless assembly of many DNA parts simultaneously or in vivo for the site-specific insertion or deletion of one or more DNA molecules into the host genome.

Table 1 below summarizes many of the advantages of the CLIC methods of the present disclosure over existing cloning and gene editing techniques.

TABLE 1
Comparison of CLIC to existing cloning and gene editing techniques.

| | | Golden Gate | Yeast | Gibson |
|---|---|---|---|---|
| | CLIC | Assembly | Assembly | Assembly |
| Scarless | + | + | + | + |
| Multipart | + | + | + | + |
| One-pot reaction | + | + | − | + |
| (i.e. fast and easy) | | | | |
| PCR amplification of | + | + | − | − |
| parts optional | | | | |
| (i.e. cheap) | | | | |
| Amenable to large library | + | + | − | − |
| construction | | | | |
| Suitable for repetitive | + | + | − | − |
| sequences | | | | |
| No sequence constraints | + | − | + | + |
| (e.g. restriction sites) | | | | |

In Vitro Sequence Editing with Cpf1

Many technologies exist for multipart DNA assembly. In some embodiments, the present disclosure teaches Golden Gate-styled modular cloning methods. The general principle of Golden Gate cloning is based on the special ability of type IIS restriction enzymes to cleave outside of their recognition site to create compatible sticky ends. When type IIS recognition sites are placed to the far 5′ and 3′ end of any DNA fragment in inverse orientation, they are removed in the cleavage process, allowing two DNA fragments flanked by compatible sequence overhangs to be ligated seamlessly in the same reaction (see for example, Engler, C., Gruetzner, R., Kandzia, R. & Marillonnet, S. “Golden gate shuffling: a one-pot DNA shuffling method based on type IIs restriction enzymes.” PLoS ONE 4, e5553 (2009); Weber, E., Engler, C., Gruetzner, R., Werner, S. & Marillonnet, S. “A Modular Cloning System for Standardized Assembly of Multigene Constructs.” PLoS ONE 6, e16765 (2011); and Chesnet, J., Dudas, M., Harris, A., Leong, L. & Madden, K. “Methods and compositions for seamless cloning of nucleic acid molecules.” Issued as U.S. Pat. No. 8,338,091).

Traditional Golden Gate techniques however, face several important cloning speed and compatibility limitations. Most type IIS restriction enzymes rely on short ˜5-7 bp unique recognition sequences to direct their DNA cleavage. The uniqueness of each enzyme's recognition sequence limits the compatibility between enzymes and cloning vectors, each of which must be engineered to include an in-frame restriction site for every planned enzyme.

Moreover, the shortness of the recognition sequences for the restriction enzymes increases the likelihood that cloned sequences will be inadvertently cleaved by the accidental presence of a restriction site within its sequence. The need to alternate enzymes and vectors to accommodate for the type IIS limitations described above is a particularly relevant consideration during high throughput operations, where one size fits all tools are normally preferred.

In some embodiments, the present disclosure overcomes the limitations of traditional Golden Gate cloning methods by teaching the CLIC modular cloning techniques using the Cpf1 CRISPR system. CLIC shares all of the benefits of Golden Gate Assembly, while eliminating the burdensome sequence constraints since the use of a CRISPR nuclease results in long (i.e. very rare) and programmable recognition sequences.

In some embodiments, the CLIC Cpf1 cloning methods of the present disclosure do not require any engineering of the DNA sequence inserts. In some embodiments, the Cpf1 cloning methods of the present disclosure produce scarless DNA assemblies.

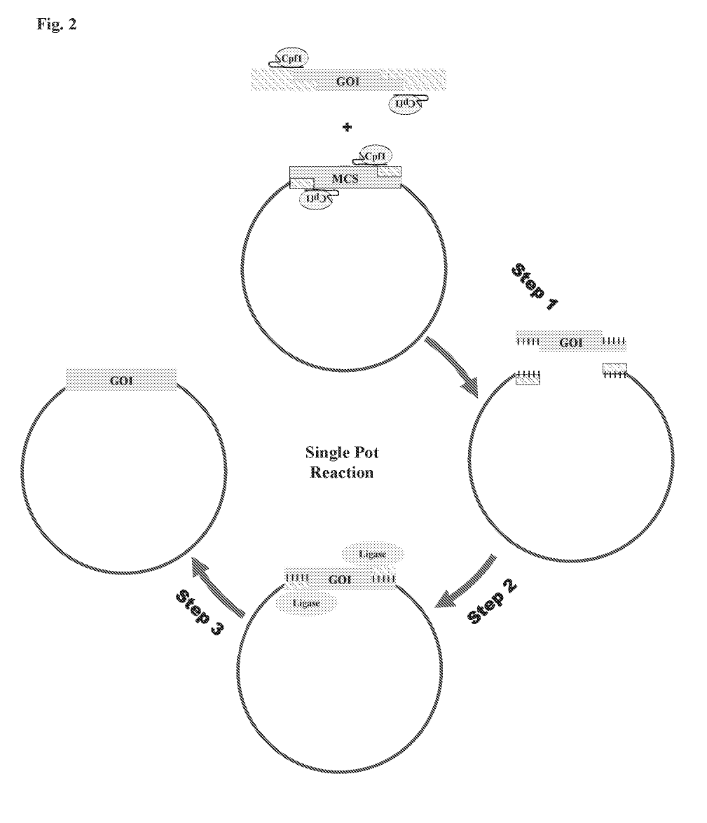

FIG. 2 depicts an embodiment of the CLIC methods of the present disclosure. In the figure, crRNA targeting polynucleotides are designed to bind in inverse orientation to the inner portion of a DNA insert region slated for deletion (e.g., a Multi Clonal Site “MCS”) so as to cleave towards the outside of the removed DNA fragment. Separate crRNA targeting polynucleotides are also designed to target the outer ends of DNA inserts (e.g., a gene of interest “GOI”), so as to remove the DNA binding sites during the reaction. In some embodiments, the crRNA guide sequences can be the same.

Designing the crRNA binding sites in inverse orientation, ensures that the sites are removed in the cleavage process, allowing two DNA fragments flanked by compatible sequence overhangs to be ligated seamlessly in the same reaction.

Compatible sticky ends from the vectors hybridize with their corresponding sticky ends in the GOI DNA. Hybridized DNA is then ligated using a ligase or other ligation method (e.g. chemical ligation).

In some embodiments, the crRNAs of the present disclosure are custom designed for each cleavage reaction. In other embodiments, standard crRNAs are designed to be reused with specific vectors and/or inserts.

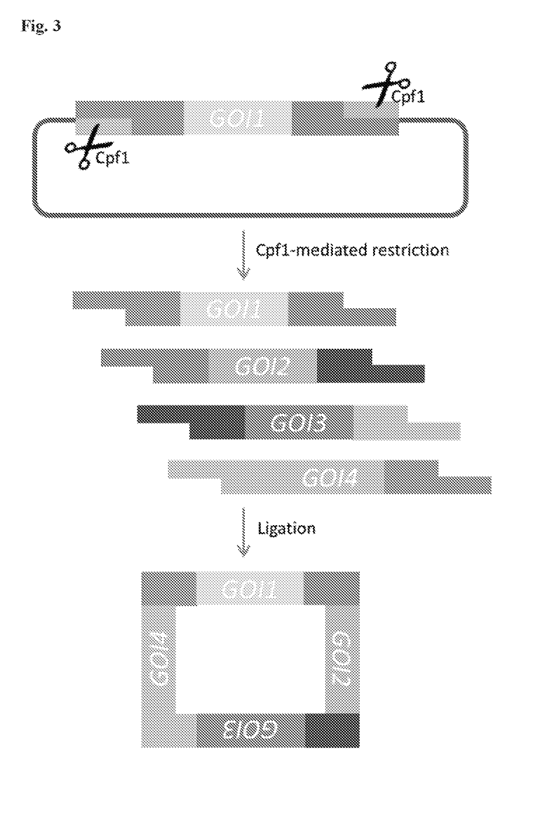

In other embodiments, the CLIC techniques of the present disclosure can be used for multi-fragment cloning. For example, FIG. 3 of the specification depicts another embodiment of the CLIC cloning methods of the present disclosure. In this figure, crRNA targeting polynucleotides are designed to target the outer ends of various GOI fragments derived from circular plasmids, or linear DNA. Each GOI DNA insert is cleaved, so as to produce a 3′ sticky end that is compatible with the 5′ end of another GOI insert. The compatible sticky ends of each GOI insert are allowed to hybridize to assemble into the final DNA molecule. Assembled DNA is ligated in the same reaction as the Cpf1 cleavage.

In some embodiments, the in vitro methods of the present disclosure are carried out by mixing previously synthesized plasmids, crRNAs, insert oligos, and Cpf1 protein.

In Vivo Sequence Editing Using Cpf1

In some embodiments, the present disclosure also teaches CLIC Cpf1 mediated methods of in vivo gene editing. In some embodiments, the CRISPR Cpf1 in vivo gene editing methods of the present disclosure do not require the presence of HDR mechanisms.

Existing techniques for targeted genome editing with CRISPR/Cas9 rely on the cell's native ability to repair double strand breaks via homologous recombination (Dicarlo, J. E. et al. “Genome engineering in Saccharomyces cerevisiae using CRISPR-Cas systems.” Nucleic Acids Res (2013). doi:10.1093/nar/gkt135). In organisms with low rates of homologous recombination, genome editing with CRISPR/Cas9 is often inefficient.

In some embodiments, CLIC gets around the aforementioned problem by supplying both the machinery for generating a double strand break at a specific location in the genome (CRISPR/Cpf1) and the machinery for repairing that double strand break in a controlled manner (DNA ligase) (see Zetsche, B. et al. 2015. “Cpf1 Is a Single RNA-Guided Endonuclease of a Class 2 CRISPR-Cas System” Cell 163, 759-771).

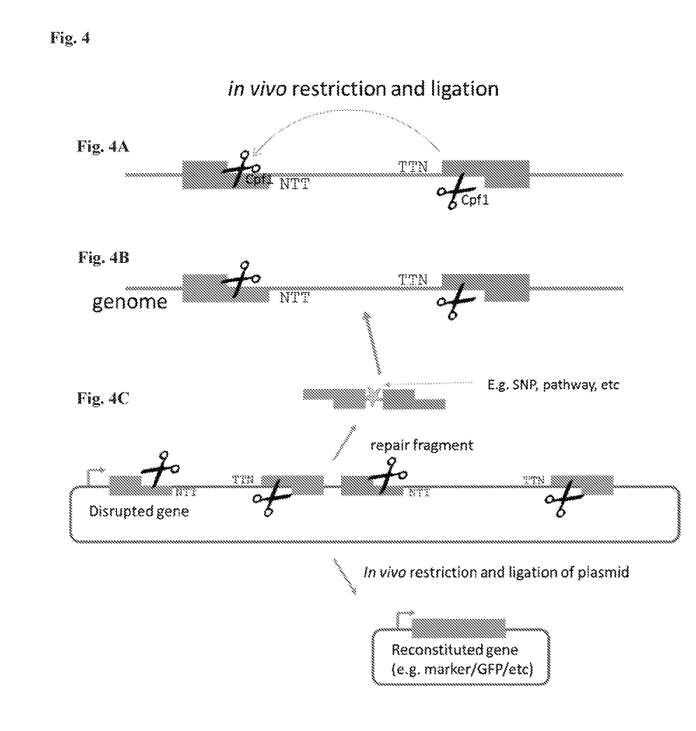

FIG. 4 of the specification depicts several embodiments of the in vivo cloning methods of the present disclosure. In some embodiments, the present disclosure teaches methods of deleting unwanted DNA regions from the genomes of engineered organisms. This process comprises targeting two Cpf1 endonucleases to locations immediately flanking the DNA region slated for deletion. The Cpf1 target sites are, in some embodiments, targeted to the inner portions of the DNA slated for deletion in an inverse orientation, such that the Cpf1 binding sites are removed by the cleavage of the target fragment. In some embodiments, the remaining sticky ends of the genomic DNA fragments created by the Cpf1 cleavage are compatible with each other, and can hybridize to each other to close the gap in the genomic DNA (FIG. 4A).

In other embodiments, the remaining sticky ends of the genomic DNA are compatible with the ends of a designed insert (FIG. 4B). In some embodiments, the sticky ends of the designed insert are produced by endonuclease reactions in vivo (e.g., via Cpf1 targeted digestions of the oligo ends within the cell). In other embodiments, the designed oligos are provided to the cell with pre-existing sticky ends (see FIG. 4C top insert fragment).

One particular embodiment of the present disclosure teaches sourcing the designed insert from an episomal plasmid in the organism (FIG. 4C). In some embodiments, the designed insert is released from the episomal plasmid by Cpf1-mediated endonuclease cleavage. In some embodiments, the episomal plasmid is designed such that removal of the designed insert reconstitutes a marker gene. Thus in some embodiment, the cells undergoing gene editing of the present disclosure can be identified by the expression of one or more marker genes.

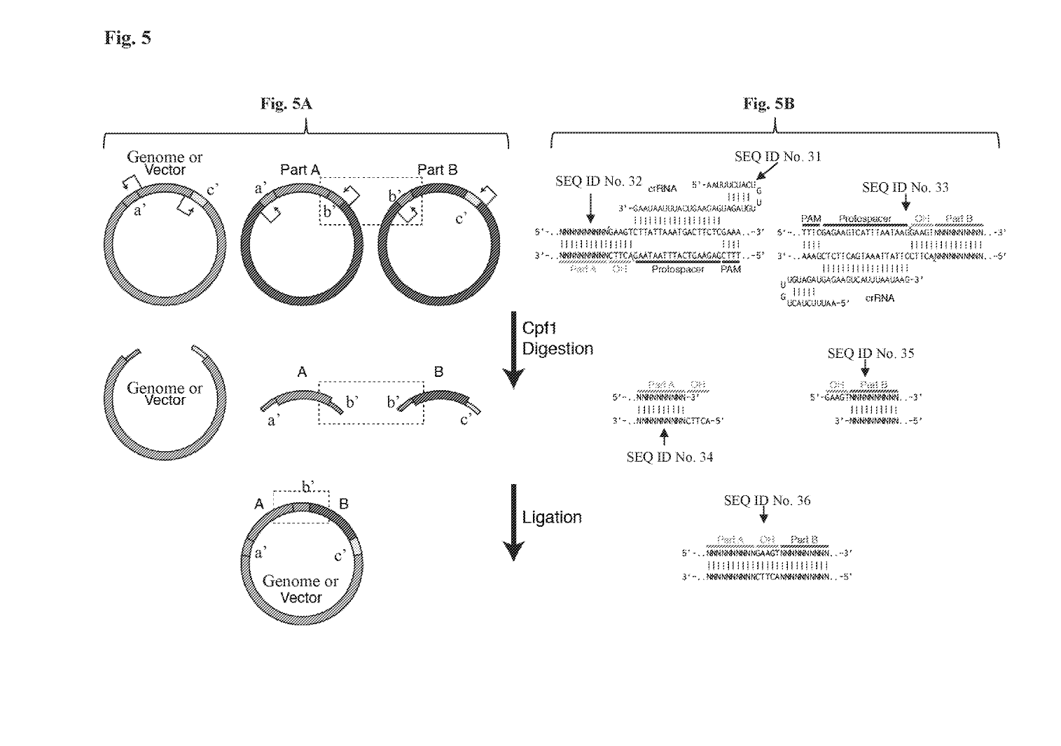

FIG. 5 of the specification depicts a CLIC method of multi-part cloning assembly in vitro or in vivo. In this figure, a vector or genome is cleaved with a Cpf1 endonuclease to create two sticky ends with distinct 5 nt overhangs a′ and c′ (FIG. 5A, top). Insert plasmids or linear PCR oligos are similarly digested by Cpf1 complexes to produce sticky ends with overhangs a′ and b′ for the Part A insert, and sticky ends with overhangs b′ and c′ for the Part B insert (FIG. 5A, top). The 3′ sticky end a′ from the vector or genome hybridizes with the compatible 5′ sticky end a′ from the Part A insert. The 3′ sticky end b′ of the Part A insert similarly hybridizes with the 5′ sticky end b′ of the Part B insert. Finally, the 3′ sticky end c′ of the Part B insert hybridizes with the 5′ c′ sticky end of the vector or genome, and the reconstituted DNA is ligated with a DNA ligase.

FIG. 5B depicts the crRNA and target sequences for the center cut of the CLIC example of FIG. 5A (see dotted lines). In this example, the crRNA sequence (SEQ ID No. 31) contains the guide sequence responsible for binding to the Part A or Part B vector, adjacent to the appropriate PAM (FIG. 5B, Top). An example sequence for the target DNA regions is provided as SEQ ID No. 32 and 33). The resulting cut creates 3′ and 5′ sticky ends for the Part A and Part B inserts respectively, with 5 nt 3′overhangs. These sequences for these sticky ends are provided as SEQ ID Nos. 34 and 35 (FIG. 5B, Middle). The resulting sticky ends hybridize according to the overhanging sequence and are ligated together (FIG. 5B, Bottom). Sequence for the ligated product provided as SEQ ID. No. 36.

In some embodiments, designed inserts of the present disclosure comprise inverted repeat sequences for looping out unwanted DNA as described in other portions of this specification. Thus, in some embodiments, the present disclosure teaches methods of inserting designed inserts into genomic regions with one or more selection markers, wherein said selection markers can later be looped out according to the methods of the present disclosure.

A person having skill in the art will recognize that the CLIC methods for in vivo genome editing of the present disclosure proceeds in much the same was as was described for the in vitro DNA assembly, except that genomic DNA takes the place of vector DNA as the recipient of the part(s) being assembled.

### Transposon-Removal Via Cpf1

In some embodiments, the present disclosure teaches methods of inactivating transposons in certain organisms. Multiple copies of the same transposon-like sequences often exist in production host organisms. These elements are known to copy and paste themselves at random integration sites throughout the genome. This is an undesirable cause of instability in production host strains, which can negatively impact strain performance and process economics. Since all copies of these elements in a genome have nearly identical sequences, they can be removed using common crRNA sequences and the editing-by-ligation strategy described above.

Thus, in some embodiments, the present disclosure teaches methods of designing and using crRNA oligos targeting one or more transposon or transposon-like sequences. In some embodiments, Cpf1 endonucleases are targeted to sequences within the transposon in inverse orientation, such that the Cpf1 binding sites are removed with the deletion of the transposon. In some embodiments, the remaining sticky ends of the cleaved genome are compatible, so as to be able to hybridize to each other and close the DNA gap.

In some embodiments, the methods of the present disclosure comprise ligating all the compatible hybridized sticky ends produced according to the Cpf1 digestions disclosed herein.

### Expression, Purification, and Delivery

In some embodiments, the present disclosure teaches methods and compositions of vectors, constructs, and nucleic acid sequences encoding the gene editing complexes of the present disclosure. In some embodiments, the present disclosure teaches plasmids or other constructs for transgenic or transient expression of the Cpf1 protein.

In some embodiments, the present disclosure teaches a plasmid encoding a chimeric Cpf1 protein comprising in-frame sequences for protein fusions of one or more of the other polypeptides described herein, including, but not limited to a ligase, a linker, and an NLS.

In some embodiments, the plasmids and vectors of the present disclosure will encode for the Cpf1 protein(s) and also encode the crRNA, and/or donor insert sequences of the present disclosure. In other embodiments, the different components of the engineered complex can be encoded in one or more distinct plasmids. In some embodiments, the present disclosure teaches extrachromosomal expression of one or more of the CLIC components. That is, in some embodiments, the present disclosure teaches extra chromosomal expression of the Cpf1 protein. In some embodiments, the present disclosure teaches extra chromosomal expression of the one or more crRNAs/guide RNAs.

In some embodiments, the plasmids/constructs of the present disclosure can be used across multiple species. In other embodiments, the plasmids/constructs of the present disclosure are tailored to the organism being transformed. In some embodiments, the sequences of the present disclosure will be codon-optimized to express in the organism whose genes are being edited. Persons having skill in the art will recognize the importance of using promoters providing adequate expression for gene editing. In some embodiments, the plasmids for different species will require different promoters.

In some embodiments, the plasmids and vectors of the present disclosure are selectively expressed in the cells of interest. Thus in some embodiments, the present application teaches the use of ectopic promoters, tissue-specific promoters, developmentally-regulated promoters, or inducible promoters. In some embodiments, the present disclosure also teaches the use of terminator sequences.

Persons skilled in the art will immediately recognize that all disclosed methods of expressing Cpf1 endonuclease is equally applicable to other CRISPR endonucleases or Targetable Enzymes.

### Transformation

In some embodiments, the present disclosure teaches the use of transformation of the plasmids and vectors disclosed herein. Persons having skill in the art will recognize that the plasmids of the present disclosure can be transformed into cells through any known system as described in other portions of this specification. For example, in some embodiments, the present disclosure teaches transformation by particle bombardment, chemical transformation, agrobacterium transformation, nano-spike transformation, and virus transformation.

In some embodiments, the vectors of the present disclosure may be introduced into the host cells using any of a variety of techniques, including transformation, transfection, transduction, viral infection, gene guns, or Ti-mediated gene transfer. Particular methods include calcium phosphate transfection, DEAE-Dextran mediated transfection, lipofection, or electroporation (Davis, L., Dibner, M., Battey, I., 1986 “Basic Methods in Molecular Biology”). Other methods of transformation include for example, lithium acetate transformation and electroporation See, e.g., Gietz et al., Nucleic Acids Res. 27:69-74 (1992); Ito et al., J. Bacterol. 153:163-168 (1983); and Becker and Guarente, Methods in Enzymology 194:182-187 (1991). In some embodiments, transformed host cells are referred to as recombinant host strains.

In some embodiments, the present disclosure teaches high throughput transformation of cells using the 96-well plate robotics platform and liquid handling machines of the present disclosure.

In some embodiments, the present disclosure teaches methods for getting exogenous protein (Cpf1 and DNA ligase), RNA (crRNA), and DNA (target DNA to be ligated into the genome) into the cell are required. Various methods for achieving this have been described previously including direct transfection of protein/RNA/DNA or DNA transformation followed by intracellular expression of RNA and protein (Dicarlo, J. E. et al. “Genome engineering in Saccharomyces cerevisiae using CRISPR-Cas systems.” Nucleic Acids Res (2013). doi:10.1093/nar/gkt135; Ren, Z. J., Baumann, R. G. & Black, L. W. “Cloning of linear DNAs in vivo by overexpressed T4 DNA ligase: construction of a T4 phage hoc gene display vector.” Gene 195, 303-311 (1997); Lin, S., Staahl, B. T., Alla, R. K. & Doudna, J. A. “Enhanced homology-directed human genome engineering by controlled timing of CRISPR/Cas9 delivery.” Elife 3, e04766 (2014)).

In some embodiments, the present disclosure teaches screening transformed cells with one or more selection markers as described above. In one such embodiment, cells transformed with a vector comprising a kanamycin resistance marker (KanR) are plated on media containing effective amounts of the kanamycin antibiotic. Colony forming units visible on kanamycin-laced media are presumed to have incorporated the vector cassette into their genome. Insertion of the desired sequences can be confirmed via PCR, restriction enzyme analysis, and/or sequencing of the relevant insertion site.

In other embodiments, a portion, or the entire complexes of the present disclosure can be delivered directly to cells. Thus, in some embodiments, the present disclosure teaches the expression and purification of the polypeptides and nucleic acids of the present disclosure. Persons having skill in the art will recognize the many ways to purify protein and nucleic acids. In some embodiments, the polypeptides can be expressed via inducible or constitutive protein production systems such as the bacterial system, yeast system, plant cell system, or animal cell systems. In some embodiments, the present disclosure also teaches the purification of proteins and or polypeptides via affinity tags, or custom antibody purifications. In other embodiments, the present disclosure also teaches methods of chemical synthesis for polynucleotides.

In some embodiments, persons having skill in the art will recognize that viral vectors or plasmids for gene expression can be used to deliver the complexes disclosed herein. Virus-like particles (VLP) can be used to encapsulate ribonucleoprotein complexes or recombinant expression, and purified ribonucleoprotein complexes disclosed herein can be purified and delivered to cells via electroporation or injection.

Persons skilled in the art will immediately recognize that the aforementioned references to vectors encoding for Cpf1 endonucleases are equally applicable to other CRISPR endonucleases or Targetable Enzymes.

### Target Sequence Selection Algorithm

In some embodiments, the present disclosure teaches algorithms designed to facilitate CRISPR target selections. In some embodiments, the software program is designed to identify candidate CRISPR target sequences on both strands of an input DNA sequence based on desired guide sequence length and a CRISPR motif sequence (PAM, protospacer adjacent motif) for a specified CRISPR enzyme. For example, target sites for Cpf1 from Francisella novicida U112, with PAM sequences TTN, may be identified by searching for 5′-TTN-3′ both on the input sequence and on the reverse-complement of the input. The target sites for Cpf1 from Lachnospiraceae bacterium and Acidaminococcus sp., with PAM sequences TTTN, may be identified by searching for 5′-TTTN-3′ both on the input sequence and on the reverse complement of the input. Likewise, target sites for Cas9 of S. thermophilus CRISPR1, with PAM sequence NNAGAAW, may be identified by searching for 5′-Nx-NNAGAAW-3′ both on the input sequence and on the reverse-complement of the input. Likewise, target sites for Cas9 of S. thermophilus CRISPR, with PAM sequence NGGNG, may be identified by searching for 5′-N, NGGNG-3′ both on the input sequence and on the reverse-complement of the input. The value “x” in Nx may be fixed by the program or specified by the user, such as 20.

In some embodiments, the algorithms of the present disclosure further facilitate the identification of compatible Cpf1 sites within open reading frames (ORFs). For example, in some embodiments, the for example, the algorithms of the present disclosure can be used to identify viable Cpf1 sites that when combined with a second site will generate compatible overhangs for enabling ligation, thereby excluding part, or the whole of the ORF

Since multiple occurrences in the genome of the DNA target site may lead to nonspecific genome editing, after identifying all potential sites, the present disclosure teaches filtering out sequences based on the number of times they appear in the relevant reference genome. For those CRISPR enzymes for which sequence specificity is determined by a ‘seed’ sequence (such as the first 5 bp of the guide sequence for Cpf1-mediated cleavage) the filtering step may also account for any seed sequence limitations.

In some embodiments, algorithmic tools can also identify potential off target sites for a particular guide sequence. For example in some embodiments Cas-Offinder can be used to identify potential off target sites for Cpf1 (see Kim et al., 2016. “Genome-wide analysis reveals specificities of Cpf1 endonucleases in human cells” published online Jun. 6, 2016).

In some embodiments, the user may be allowed to choose the length of the seed sequence. The user may also be allowed to specify the number of occurrences of the seed:PAM sequence in a genome for purposes of passing the filter. The default is to screen for unique sequences. Filtration level is altered by changing both the length of the seed sequence and the number of occurrences of the sequence in the genome. The program may in addition or alternatively provide the sequence of a guide sequence complementary to the reported target sequence(s) by providing the reverse complement of the identified target sequence(s).

Persons having skill in the art would similarly be able to identify target sites for Target Enzymes of the present disclosure.

### Kits

In some embodiments, the disclosure provides kits containing any one or more of the elements disclosed in the above methods and compositions. In some embodiments, the kit comprises a vector system and instructions for using the kit. In some embodiments, the vector system comprises (a) a first regulatory element operably linked to a polynucleotide encoding for a crRNA/guide RNA sequence, said polynucleotide comprising one or more insertion sites for inserting a desired guide sequence downstream of the loop portion of the crRNA, wherein when expressed, the crRNA sequence directs sequence-specific binding of a CRISPR Cpf1 complex to a target sequence in an engineered cell. In some embodiments, the vector system further contains a (b) second regulatory element operably linked to an enzyme-coding sequence encoding a CRISPR Cpf1 enzyme. In some embodiments, the vectors system further comprises a (c) third regulatory element operably linked to a polynucleotide encoding a functional ligase. In some embodiments, the CRISPR Cpf1 endonuclease of the kit is a chimeric Cpf1 comprising an NLS, and/or a ligase as described above.

Elements may be provided individually or in combinations, and may be provided in any suitable container, such as a vial, a bottle, or a tube. In some embodiments, the kit includes instructions in one or more languages, for example in more than one language.

In some embodiments, a kit comprises one or more reagents for use in a process utilizing one or more of the elements described herein (e.g., purified Cpf1 endonuclease). Reagents may be provided in any suitable container. For example, a kit may provide one or more reaction or storage buffers. Reagents may be provided in a form that is usable in a particular assay, or in a form that requires addition of one or more other components before use (e.g. in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10. In some embodiments, the kit comprises one or more oligonucleotides corresponding to a crRNA sequence for insertion into a vector so as to operably link the crRNA sequence and a regulatory element.

Persons skilled in the art will immediately recognize that the aforementioned disclosure of kits comprising Cpf1 endonuclease are equally applicable to other CRISPR endonucleases or Targetable Enzymes.

### EXAMPLES

The following examples are given for the purpose of illustrating various embodiments of the disclosure and are not meant to limit the present disclosure in any fashion. Changes therein and other uses which are encompassed within the spirit of the disclosure, as defined by the scope of the claims, will occur to those skilled in the art.

### Example 1: Production of Purified Cpf1 Protein

Cpf1 protein was purified from bacterial cultures for use in future in vitro CLIC reactions. The coding sequence for the FnCpf1 was cloned into a standard bacterial expression pD454-HMBp based backbone vector (pUC ori. AmpR, T7 promoter (IPTG inducible, His-tag. MBP fusion, TEV protease cleavage site) and was transformed into a E. coli BL21(DE3) protein production host. The transformed cultures were grown in standard bacterial media and were induced with IPTG. Cultures were then lysed, and the resulting protein extractions were nickel purified, followed by the removal of tags with TEV protease.

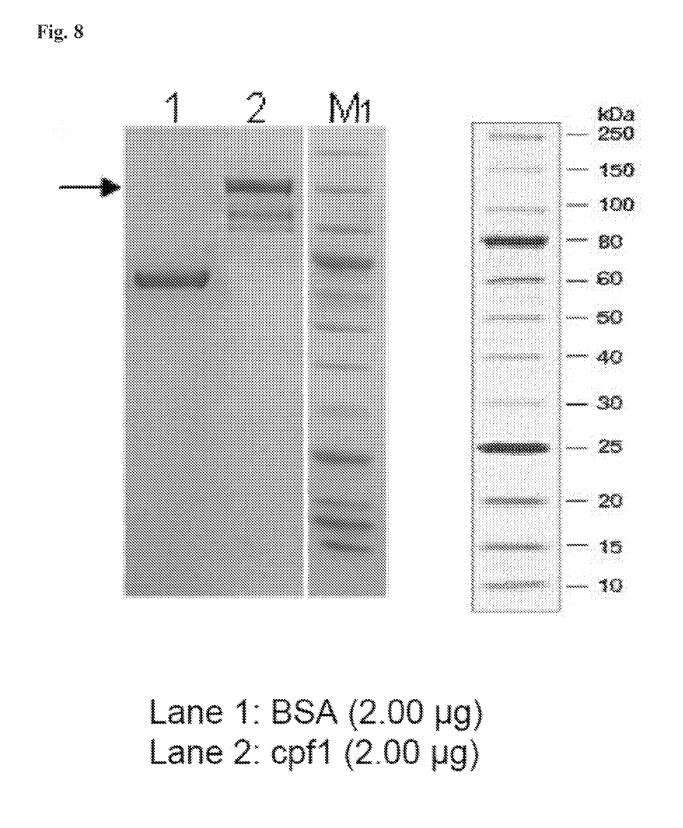

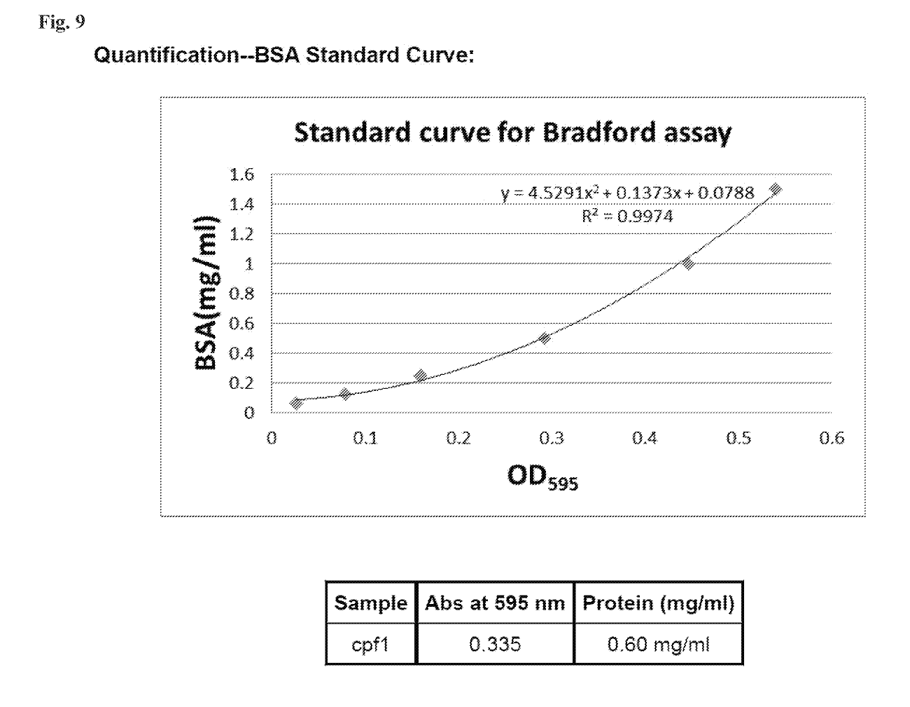

Purified Cpf1 protein was visualized in a SDS-PAGE gel to confirm purity (see lane 2 in FIG. 8). Cpf1 protein concentration was determined via standard Bradford Assay quantification methods (see FIG. 9).

### Example 2: Cpf1 Mediated Digestion and Ligation

Purified Cpf1 enzyme from Example 1 was incubated with a 1956 bp PCR fragment and a crRNA to test for Cpf1-mediated digestion. The 1956 bp PCR sequence for the reaction was derived from a PCR an amplification of pWD031 plasmid, resulting in a PCR product as disclosed in SEQ ID NO. 79. The crRNA was derived from an in vitro transcription of a linear DNA template using a T7 HiScribe® RNA synthesis kit, resulting in a crRNA with the sequence disclosed in SEQ ID NO. 85.

The crRNA sequence was designed such that successful Cpf1 cleavage of the 1956 bp PCR fragment would result in a 1500 bp and a 500 bp fragment (SEQ ID NO. 84, and SEQ ID NO. 83, respectively). A first reaction was allowed to digest the PCR fragment for 20 minutes at 37 degrees Celsius to confirm Cpf1 activity. A second reaction was allowed to digest the PCR fragment for 20 minutes at 37 Celsius, followed by a heat inactivation of the Cpf1 enzyme, and a 2-hour incubation with T7 DNA ligase in T4 DNA ligase buffer at room temperature. The reactions were run on a standard agarose gel and the resulting DNA fragments were analyzed.

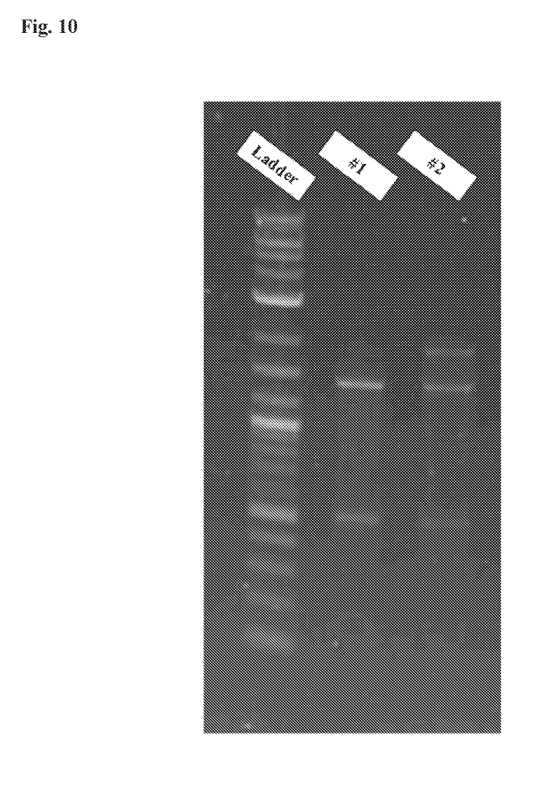

The Cpf1-digested reaction exhibited the expected 1500 bp and 500 bp fragments. The ligase-incubated reaction exhibited the digestion fragments, but also showed a significant band at 1956 bp, representing the re-ligated PCR product (FIG. 10).

### Example 3: CLIC In Vitro Single Pot Cloning with Cpf1-Fragment Ligation

In order to test Cpf1's ability to conduct single-pot in vitro DNA assembly, a two fragment digestion/ligation reaction was conducted. Two PCR products with sequences disclosed in SEQ ID No. 86 and 87 were combined in a Cpf1 reaction with a pre-synthesized crRNAs 1 and 3 with the sequence disclosed in SEQ ID No. 85 and 88.

The crRNA sequences were designed so as to direct the Cpf1 nuclease to the outer portions of the PCR products, such that the Cpf1 binding sites would be removed once the reaction was complete. The Cpf1 complex was thus designed to be in an inverse orientation to ensure that digested PCR products would cease to be Cpf1 substrates, and would thus be available for subsequent ligation steps of the experiment. The reaction also included a T7 ligase purchased from commercial vendors. A control reaction for this experiment omitted the ligase, but was otherwise identical. Both reactions were conducted using a T4 ligase buffer.

Rather than incubating the reaction at 37 degrees Celsius (the optimum temperature for the Cpf1 enzyme), the reaction was cycled between 37 Celsius for two minutes, and 20 Celsius (the optimum ligase temperature) for five minutes for 25 cycles to allow for ligase activity between bursts of digestion. The resulting products were run on a standard agarose gel with a DNA ladder.

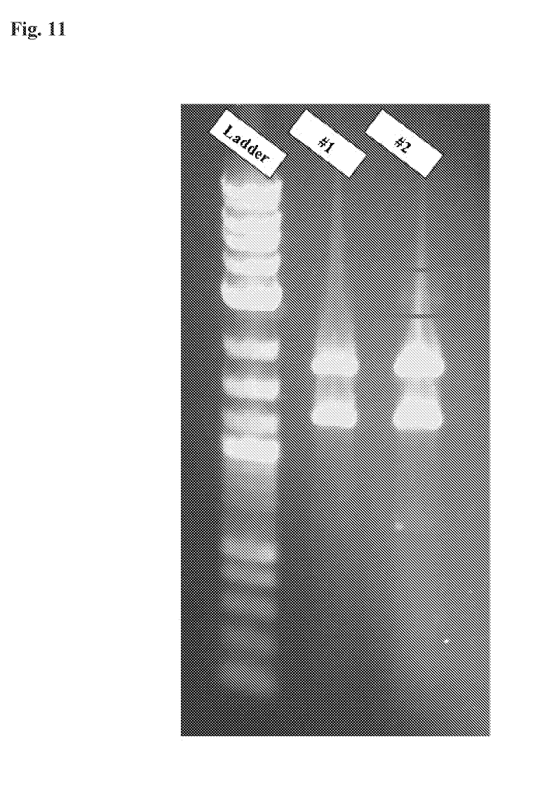

FIG. 11 shows the resulting bands from the CLIC reaction. Control lane 1 included two bands corresponding to the digested ˜1300 bp and ˜1800 bp PCR fragments corresponding to digested SEQ ID NOs. 85 and 88. Ligase experimental lane 2 includes a visible band of ˜3000 bp, corresponding to the CLIC ligation of the two Cpf1 digested PCR products.

This experiment thus demonstrated the ability of Cpf1 to be used for single-pot CLIC cloning reactions.

### Example 4: In Vivo Cpf1 Cleavage

An in vivo CLIC digestion reaction was conducted, in order to validate Cpf1 endonuclease activity in living hosts. The Cpf1 coding sequence from Example 1 was re-cloned into a standard bacterial expression vector with the plasmid sequence as disclosed in SEQ ID No. 29. The Cpf1 expression vector further comprised a crRNA expression cassette with the targeting guide sequence disclosed in SEQ ID NO. 30 (shown in DNA form).

Two additional “resistance” plasmids were cloned, each containing a Kanamycin resistance marker. One of the resistance plasmids was designed to be a perfect Wild Type target for the crRNA of the Cpf1 plasmid (e.g. designed to have a validated CRISPR landing site for the CRISPR complex disclosed above). The second resistance plasmid contained a Mutant PAM designed to reduce Cpf1 cleavage of the target. Sequences for both resistance plasmids are disclosed as SEQ ID No. 80 (Wild Type PAM) and SEQ ID No. 81 (Mutant PAM).

E. coli cells were transformed with the cloned vectors according to four experimental treatments: 1) Wild Type PAM resistance vector, 2) Wild Type PAM resistance vector with the co-transformed Cpf1/crRNA vector, 3) Mutant PAM resistance vector, and 4) Mutant PAM resistance vector with the co-transformed Cpf1/crRNA vector. Transformed cells were plated on media containing the resistance selection marker, such that only cells comprising intact resistance plasmids would survive.

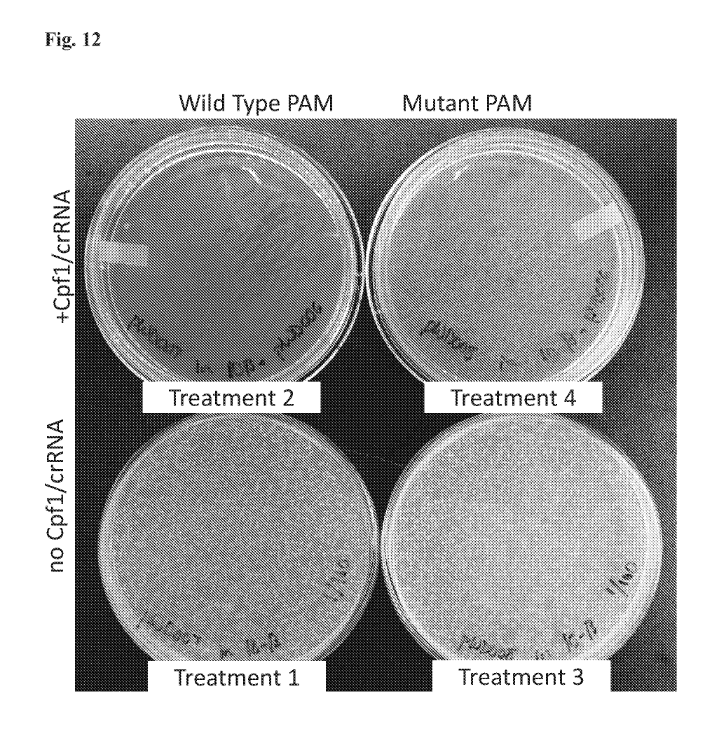

FIG. 12 depicts the results of the experiment. Cells from Treatment 2, transformed with both the Cpf1/crRNA vector and the Wild Type resistance plasmid showed a marked decrease in colony forming units compared to Treatment 1 plates containing only the Wild Type resistance plasmid. In contrast, cells from Treatment 4, transformed with both the Cpf1/crRNA vector and the Mutant Pam showed little difference in the number of colony forming units compared to Treatment 3 plates containing the Mutant PAM plasmid.

Cpf1 co-expression successfully targeted and disabled Wild Type resistance plasmids in vivo. This effect could be reversed as Cpf1 cleavage of target plasmids was thwarted by mutating the PAM sequence.

### Example 5: In Vivo Gene Editing with Cpf1

CLIC DNA assemblies will be validated in in vitro gene editing experiments. Briefly, engineered Escherichia coli strains chromosomally expressing either T4 or T7 ligase genes, and FnCpf1 genes will be transiently transformed with extrachromosomal plasmids expressing CRISPR arrays encoding crRNAs targeting various genes of interest. Initial gene targets will include (but will not necessarily be limited to) yhfS and upp.

The crRNAs for this example will be targeted to two compatible locations flanking each target gene, in order to induce a deletion a portion, or the entire gene ORF. The crRNAs would be further designed to position the Cpf1 endonuclease on either side of the gene ORF in an outwardly facing inverse orientation, according to the CLIC methods of the present disclosure. Control bacterium would include crRNAs designed to position the Cpf1 endonuclease such that one, or both of the crRNA target locations was oriented to face inward towards the deletion.

Transformed E. coli would be screened to determine deletion rates for the targeted gene. For example, disruption of the upp gene will be determined by screening for bacteria that becomes insensitive to 5-fluorouracil exposure.

Successful transformants will then be rehabilitated using CLIC in vivo DNA insertions. For example, bacteria with disrupted upp genes will be repaired by re-inserting the upp gene into the mutated locus, without the addition of any scars.

Briefly, malfunctioning E. coli strains will be transformed with extrachromosomal CRISPR arrays encoding crRNAs that are designed to position the Cpf1 endonuclease on either side of the disrupted ORF in an outwardly facing inverse orientation, according to the CLIC methods of the present disclosure. Control bacterium would include crRNAs designed to position the Cpf1 endonuclease such that one, or both of the crRNA target locations was oriented to face inward towards the deletion.

Insertion sequences will be provided as either pre-processed oligos with pre-existing staggered cuts (e.g., hybridized staggered oligos with protected ends, such as with phosphorothioate nucleotides), or could also be provided as linear or circular inserts sequences for in vivo processing. In the latter, the insert DNA will be designed to include the target sequences of one or both of the crRNAs targeted to the genome, except that the target sites will be oriented such that the Cpf1 endonuclease was oriented to face inward towards the insert in an inverse orientation.

Rehabilitated bacteria will be screened via similar methods as described above. For example, bacterial cultures will be exposed to ethionine to identify return to wild type sensitivity. Alternatively, the insert will also include a selection marker to facilitate screening.

### Example 6: Transposon Inactivation with Cpf1

Transposon inactivation methods of the present disclosure will also be validated as described in Example 6. Briefly, engineered Escherichia coli strains chromosomally expressing either T4 or T7 ligase genes, and FnCpf1 genes will be transiently transformed with extrachromosomal plasmids expressing CRISPR arrays encoding crRNAs targeting selected transposon sequences.

The crRNAs for this example will be targeted to two compatible locations flanking the selected transposon, in order to induce its deletion from the genome. Initial trials will target transposons with multiple copies with high sequence similarity. The crRNAs for this experiment would be further designed to position the Cpf1 endonuclease on either side of the transposon element in an outwardly facing inverse orientation, according to the CLIC methods of the present disclosure.

Successful events will be identified via PCR screens of selected transposon events, much like the identification of T-DNAs.

### Example 7: Identification of Additional Cpf1 Gene Homologs and Orthologs

The Cpf1 polypeptide sequence from Francisella tularensis subsp. Novicida U112 disclosed in SEQ ID NO: 7 was used to identify additional putative Cpf1 homologs and orthologs from other eukaryotic and prokaryotic organisms.

Briefly, the amino acid sequence of SEQ ID NO: 7 was used as the search string in the NCBI BLASTP® database to identify related sequences with high homology to the search gene. Searches were conducted with default search parameters in order to identify highly related bacterial homologs for each searched gene.

The following Table 2 provides the NCBI Reference Sequence Name of the polypeptide sequences of genes identified during this search. Additional homologs and orthologs are identifiable by additional sequence searches based on the Cpf1 sequences of the present disclosure, including those of SEQ ID Nos: 7, 8, 9, 10, 11, 12, 13, 14, 15, 16, 17, 18, 19, 20, 21, 22, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, and 78.

TABLE 2
Selected Cpf1 Gene Homologs Identified Through BLASTP ® Homology Search
Engine

| | | | |
|---|---|---|---|
| AJI156734 | KGF47514 | WP_016301126 | WP_039658684 |
| AKG06878 | KKP36646 | WP_018359861 | WP_044110123 |
| AKG08867 | KKQ36153 | WP_020988726 | WP_044910712 |
| AKG14689 | KKQ38174 | WP_021736722 | WP_044910713 |
| AKG18099 | KKR91555 | WP_023936172 | WP_044919442 |
| CDA41776 | KKT48220 | WP_023941260 | WP_045971446 |
| CDF09621 | KKT50231 | WP_024988992 | WP_046328599 |
| CDF12615 | KUJ74576 | WP_027109509 | WP_048112740 |
| CUM80100 | KXB38146 | WP_027216152 | WP_049895985 |
| CUO47728 | WP_003034647 | WP_027407524 | WP_050786240 |
| CUO57667 | WP_003040289 | WP_028248456 | WP_051666128 |
| CUO70892 | WP_004339290 | WP_028830240 | WP_052585281 |
| CUP14506 | WP_005398606 | WP_029392401 | WP_052943011 |
| CUQ77205 | WP_006283774 | WP_031492824 | WP_059369505 |
| CUQ81832 | WP_009217842 | WP_035798880 | WP_062376669 |
| EFL46285 | WP_012739647 | WP_036388671 | |
| EKE06926 | WP_013282991 | WP_036851563 | |
| EKE28449 | WP_014085038 | WP_036887416 | |
| WP_062499108 | WP_014550095 | WP_036890108 | |
| KFO67989 | WP_015504779 | WP_037975888 | |

### Example 8: In Vitro Cpf1 Assembly

This example was designed to demonstrate the flexibility of CRISPR cloning. As an initial step, several resistance plasmids encoding for Kanamycin or Chloramphenicol resistance genes were created from source vectors pzHR039 (SEQ ID No: 89) and 13000223370 (SEQ ID No:90), respectively. The Kanamycin resistance plasmids were each designed so as to include various Cpf1 landing sites flanking the GFP gene (when digested, these plasmids produce “the kanamycin resistant plasmid backbone”). The Chloramphenicol resistance plasmids were each designed so as to include various Cpf1 landing sites flanking the Chloramphenicol resistance gene (when digested, these plasmids produce “the chloramphenicol resistant insert”). Sequences, and vector maps for each plasmid used in this Example are disclosed in Table 3.

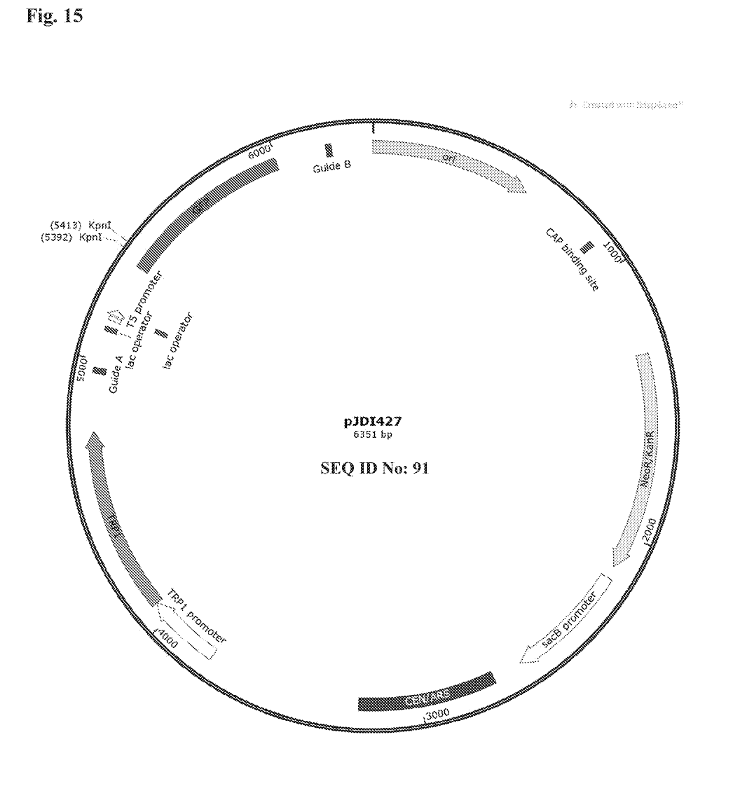

Each Kanamycin and Chloramphenicol resistant plasmid was initially linearized with type-II restriction enzymes KpnI-HF and PvuI-HF, respectively (both commercially available from NEB). The location of the KpnI and PvuI restriction sites on each plasmid are noted in the vector maps provided in FIGS. 15-22. After linearization, the resistance plasmids were no longer capable of self-replication in a bacterial host system.

Linearized resistance plasmids were then mixed with a pre-incubated mixture of 15 ug (1.58 uM final concentration) of Cpf1 enzyme and 2 uL of 5 uM of each guide RNA described below (0.167 uM final concentration) in a 60 uL reaction to form active CRISPR complexes.

The Cpf1 enzyme used in this Example was commercially obtained from IDT. The Cpf1 was sourced from Acidaminococcus sp. Cpf1 (AsCpf1). The enzyme was further modified to comprise 1 N-terminal nuclear localization sequence (NLS) and 1 C-terminal NLSs, as well as 3 N-terminal FLAG tags and a C-terminal 6-His tag.

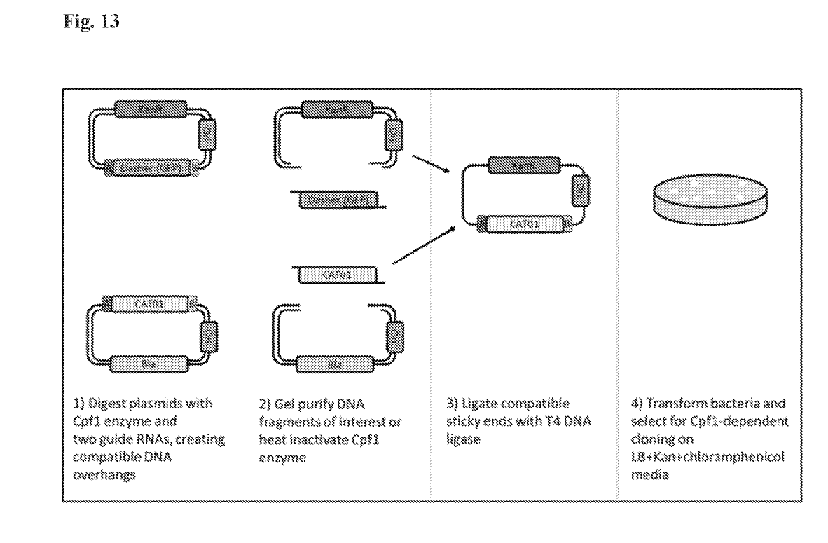

The guide RNAs used in this example were custom ordered from IDT. Each guide RNA was designed to target a different CRISPR landing site located within the linearized resistance plasmid. In this Example, the Cpf1 landing sites of the backbone plasmid were arranged in an inward orientation, such that the landing sites would remain on the vector after digestion. Table 3 provides the guide sequence portion of each guide RNA used in their DNA format (see guide sequences A-D on Table 3). The CRISPR complexes in the mixture were thus designed to cleave out the GFP gene from each kanamycin resistant plasmid to generate kanamycin resistant plasmid backbones (see FIG. 13, second panel). The CRISPR complexes in the mixture were also designed to cleave out the chloramphenicol resistance gene from the chloramphenicol resistance plasmid to generate chloramphenicol resistant inserts (see FIG. 13, second panel). The kanamycin resistant plasmid backbone and the chloramphenicol resistant insert of each reaction were similarly designed to generate compatible sticky 5′ and 3′ ends that would result in hybridization of the ends to produce a “dual resistant” kanamycin and chloramphenicol plasmid.

The linearized resistance plasmid mixtures comprising the Cpf1 and guide RNAs were allowed to incubate for 3 hours at 37 Celsius in the manufacturer's recommended Cpf1 buffer. Selected reactions were run on agarose gels and the resulting fragments were purified using standard DNA extraction kits (Zymo Research kit, used according to manufacturer's instructions). Purified (control) and unpurified (test)

DNA fragments comprising the kanamycin resistant plasmid backbone and the chloramphenicol resistant insert, each comprising two compatible Cpf1 sticky ends were combined in a new reactions with or without a T4 DNA ligase (commercially available form NEB) and transformed into NEB10-B cells (commercially available from NEB). Transformed cells were plated on media augmented with both Kanamycin and Chloramphenicol designed to prevent the growth of any cells that did not contain functional resistance plasmids.

Individual colonies were sent for sequencing to confirm junctions of Cpf1 cloning. Recovered colonies were also validated via PCR using primers described in Table 3. FIG. 13 illustrates the general experimental design described above, except that the plasmids were linearized prior to Cpf1 digestion, as described above.

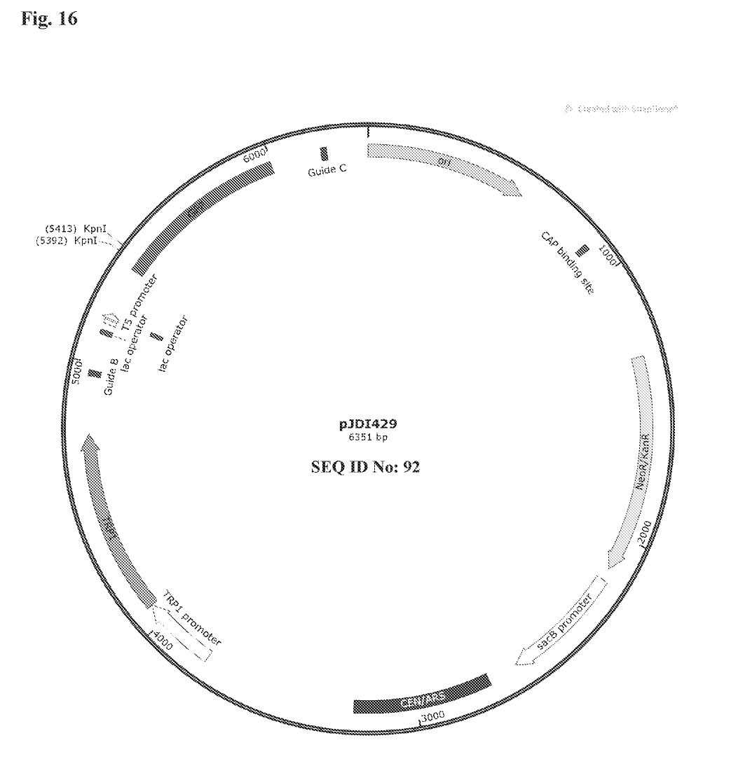

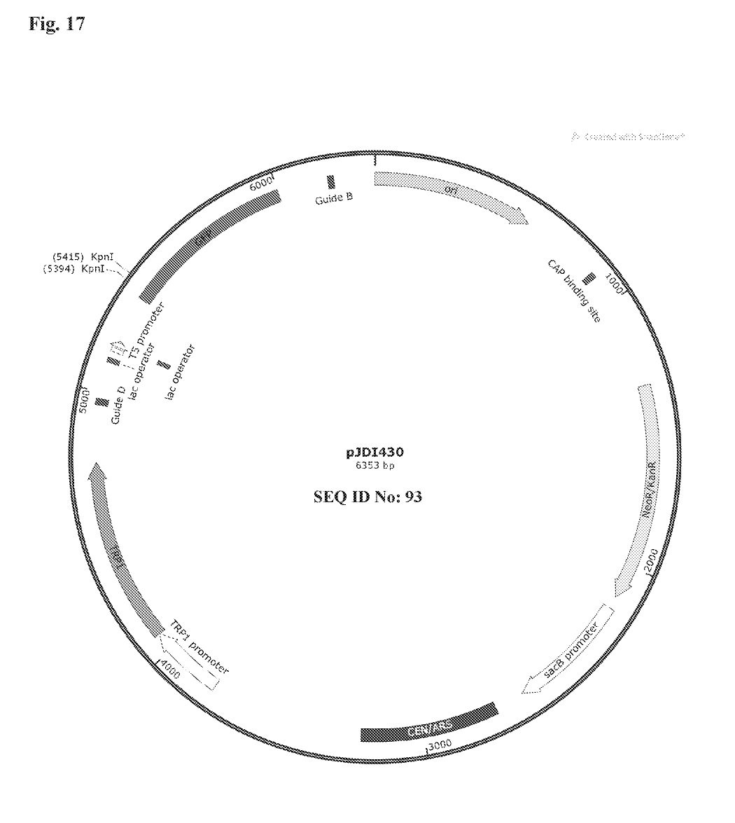

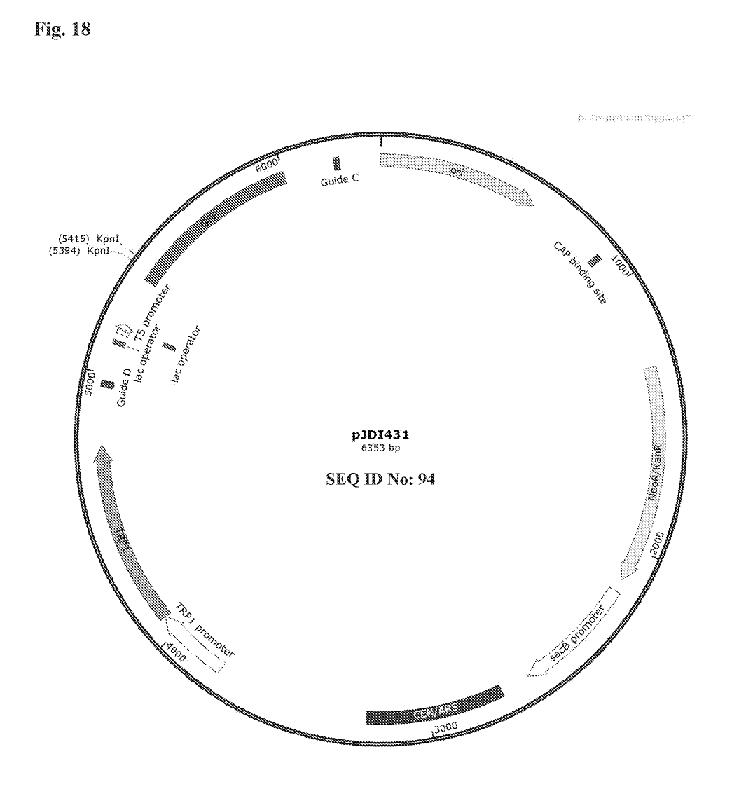

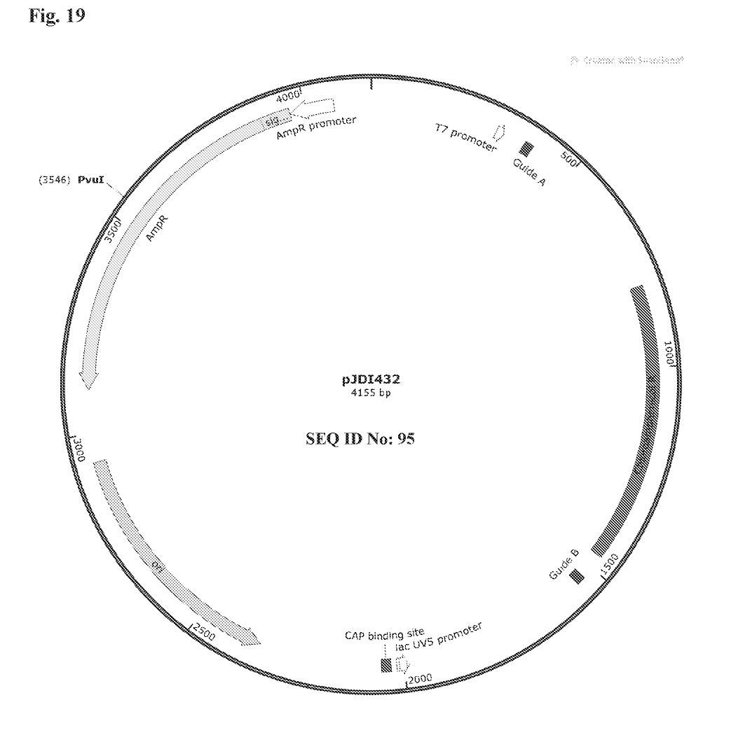

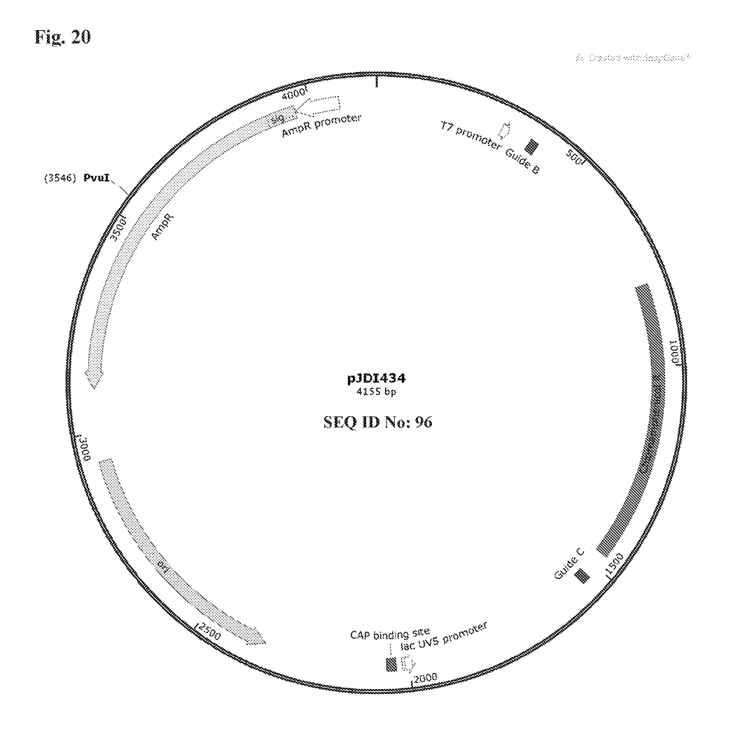

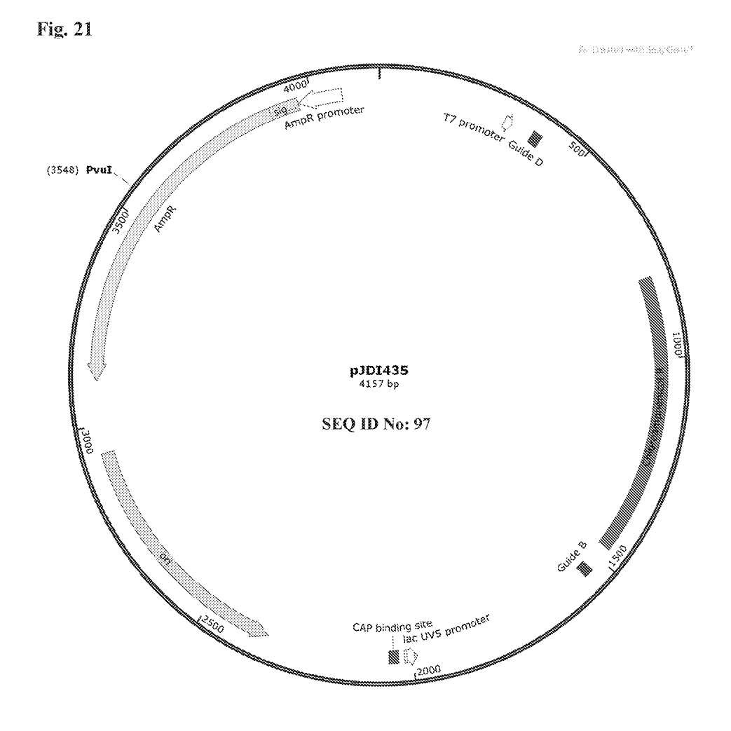

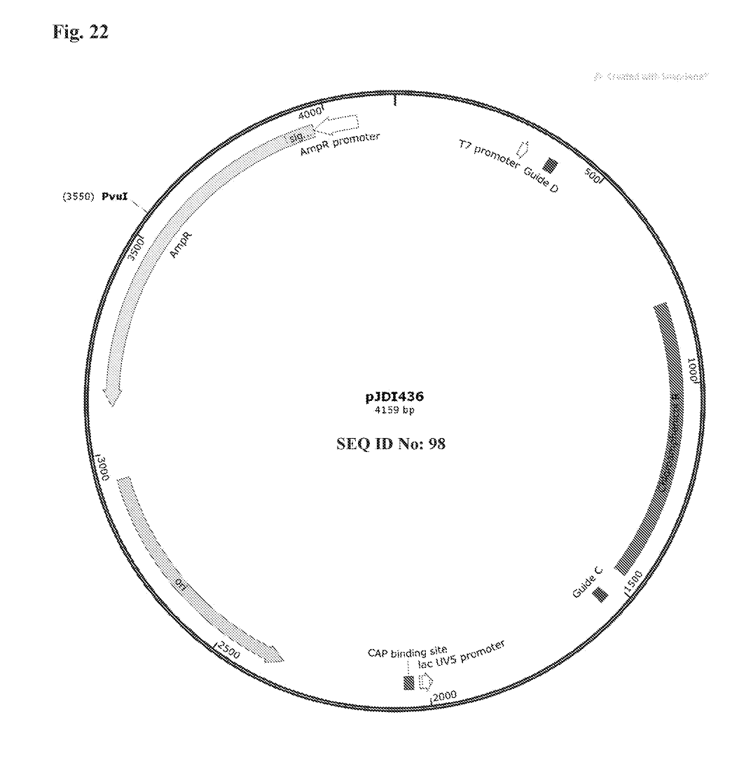

TABLE 3
List of sequences used in this Example

| Component | Description | SEQ ID NO |
|---|---|---|
| GFP Cpf1 A fwd | 5′ CAGCACCTGGATTACCCTGTTATCCCTAGTTTTGGGTTAAAGA | SEQ ID No: 99 |
| | TGGTTAAATGATTCGAAAATAATAAAGGGAAAATCA 3′ | |
| GFP Cpf1 B fwd | 5′ CAGCACCTGGATTACCCTGTTATCCCTAGTTTTGGGATGTTAA | SEQ ID No: 100 |
| | GAGTCCCTATCTTCGAAAATAATAAAGGGAAAATCA 3′ | |
| GFP Cpf1 D fwd | 5′ CAGCACCTGGATTACCCTGTTATCCCTAGTTTTGAGGAGTGTT | SEQ ID No: 101 |
| | CAGTCTCCGTGAACTCGAAAATAATAAAGGGAAAATCA 3′ | |
| GFP Cpf1 C rvs | 5′ CGCTTCCTCCTGAAAATGCAGCTAGGTAGTTTTGACCGCCCCC | SEQ ID No: 102 |
| | CCCATACCCCAATCGACATGCCGAACTCAGAAGTGA 3′ | |
| GFP Cpf1 B rvs | 5′ CGCTTCCTCCTGAAAATGCAGCTAGGTAGTTTTGGGATGTTAA | SEQ ID No: 103 |
| | GAGTCCCTATCTTCGACATGCCGAACTCAGAAGTGA 3′ | |
| CAT01 Cpf1 A fwd | 5′ TTTGGGTTAAAGATGGTTAAATGATTCGACATACACATAA | SEQ ID No: 104 |
| | AGTAGCTTGCG 3′ | |
| CAT01 Cpf1 B fwd | 5′ TTTGGGATGTTAAGAGTCCCTATCTTCGACATACACATAAA | SEQ ID No: 105 |
| | GTAGCTTGCG 3′ | |
| CAT01 Cpf1 D fwd | 5′ TTTGAGGAGTGTTCAGTCTCCGTGAACTCGACATACACATA | SEQ ID No: 106 |
| | AAGTAGCTTGCG 3′ | |
| CAT01 Cpf1 B rvs | 5′ TTTGGGATGTTAAGAGTCCCTATCTTCGACTGGAAGGACA | SEQ ID No: 107 |
| | AGGGGGACC 3′ | |
| CAT01 Cpf1 C rvs | 5′ TTTGACCGCCCCCCCCATACCCCAATCGACTGGAAGGACA | SEQ ID No: 108 |
| | AGGGGGACC 3′ | |
| Cpf1 guide | 5′ GGTTAAAGATGGTTAAATGAT 3′ | SEQ ID No: 109 |
| sequence A | | |
| Cpf1 guide | 5′ GGATGTTAAGAGTCCCTATCT 3′ | SEQ ID No: 110 |
| sequence B | | |
| Cpf1 guide | 5′ ACCGCCCCCCCCATACCCCAA 3′ | SEQ ID No: 111 |
| sequence C | | |
| Cpf1 guide | 5′ AGGAGTGTTCAGTCTCCGTGAAC 3′ | SEQ ID No: 112 |
| sequence D | | |
| pzHR039 | Source for Kanamycin resistance and GFP. See listing for full | SEQ ID NO: 89 |
| | sequence | |
| 13000223370 | Source for Chloramphenicol resistance and GFP. See listing | SEQ ID NO: 90 |
| | for full sequence | |
| pJDI427 | GFP Cpf1 AB #1a pMB1 KanR CENARS TRP1 see listing for full | SEQ ID NO: 91 |
| | sequence | FIG 15 |
| pJDI429 | GFP Cpf1 BC 3a pMB1 KanR CENARS TRP1 see listing for full | SEQ ID NO: 92 |
| | sequence | FIG 16 |
| pJDI430 | GFP Cpf1 DB 4a pMB1 KanR CENARS TRP1 see listing for full | SEQ ID NO: 93 |
| | sequence | FIG 17 |
| pJDI431 | GFP Cpf1 DC 5a pMB1 KanR CENARS TRP1 see listing for full | SEQ ID NO: 94 |
| | sequence | FIG 18 |
| pJDI432 | pJET AmpR CmR blunt Cpf1 AB 1c see listing for full sequence | SEQ ID NO: 95 |
| | | FIG 19 |
| pJDI434 | pJET AmpR CmR blunt Cpf1 BC 3a see listing for full sequence | SEQ ID NO: 96 |
| | | FIG 20 |
| pJDI435 | pJET AmpR CmR blunt Cpf1 DB 4a see listing for full sequence | SEQ ID NO: 97 |
| | | FIG 21 |
| pJDI436 | pJET AmpR CmR blunt Cpf1 DC 5d see listing for full sequence | SEQ ID NO: 98 |
| | | FIG 22 |

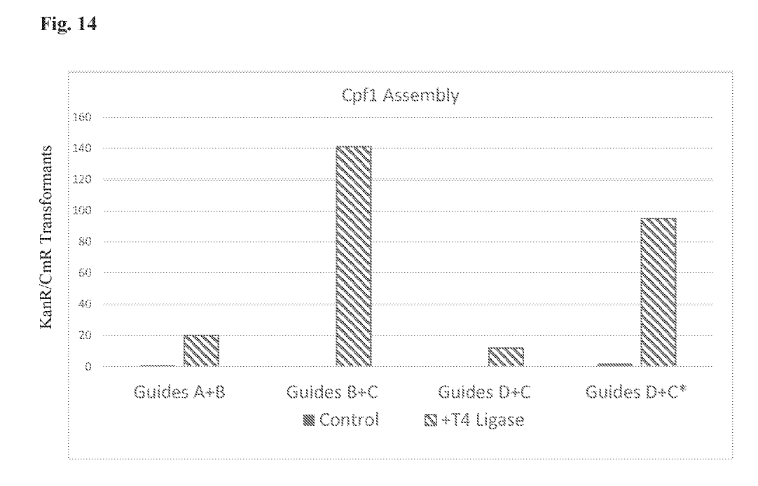

The results of this experiment are shown in Table 4 and FIG. 14. Reaction numbers for each transformation are shown along the top row, with guide RNAs used listed along the left-hand column of Table 4. The comparison of identical Cpf1 reactions with and without ligase showed a 9.9-fold increase in transformants in the presence of ligase enzyme, indicating that colony growth was due to formation of the double kanamycin and chloramphenicol resistant plasmid after Cpf1 digestion. The no-ligase reactions are matched controls designed to establish that the reactions are specific, and were not simply due to the presence of contaminating levels of undigested resistance plasmids.

Sixteen individual colonies were Sanger sequenced to verify both the upstream and downstream cloning junctions. In seven of seven upstream sequenced junctions, and eight of nine downstream junctions, the Cpf1 mediated clones from the reactions with T4 DNA ligase indicated faithful digestion and ligation.

Reactions 71 and 72 were transformed with Cpf1 digested plasmids that were not subjected to DNA gel purification steps. Cpf1 enzyme however was heat inactivated according to supplier's instructions before addition of T4 DNA ligase (reaction 72). Reactions 71 and 72 exhibited the same ligase-dependency.

TABLE 4
Resistant Transformant Colonies Comprising Cpf1-edited vectors

| | 55 | 56 | 59 | 60 | 67 | 68 | 71* | 72* |
|---|---|---|---|---|---|---|---|---|
| Guides A + B | yes | yes | | | | | | |
| Guides B + C | | | yes | yes | | | | |
| Guides D + C | | | | | yes | yes | yes | yes |
| T4 Ligase | No | Yes | No | Yes | No | Yes | No | Yes |
| # of transformants | 1 | 20 | 0 | 141 | 0 | 12 | 2 | 95 |
| (Kan Resistant | | | | | | | | |
| Colonies) | | | | | | | | |

*Plates 71 and 72 were transformed with digested DNA that had not undergone DNA gel purification after Cpf1 digestion.

### Further Embodiments of the Invention

Other subject matter contemplated by the present disclosure is set out in the following numbered embodiments:

1. A method for assembling gene constructs in vitro from a plurality of DNA fragments, said method comprising the steps of:
(a) providing a plurality of DNA fragments comprising a first and second DNA fragment, wherein said first DNA fragment comprises a sequence overlap of at least three nucleic acids anywhere within the second DNA fragment;
(b) digesting the first DNA fragment with a Cpf1 CRISPR complex, thereby creating a sticky DNA end at the 5′ and/or 3′ of said first DNA fragment, wherein said digested first DNA fragment ceases to be a target for said Cpf1 CRISPR complex;
(c) annealing the sticky end of the digested first DNA fragment from step (b) to a second compatible sticky end on the second DNA fragment; and
(d) ligating the annealed DNA fragments from step (c) together, resulting in a ligated product;
   wherein the resulting ligated product is an assembled construct.
   2. The method of embodiment 1, wherein no genetic scars are introduced into the assembled construct from practicing the method.
   3. The method of embodiment 1 or 2, wherein the Cpf1 CRISPR complex comprises i) a Cpf1 endonuclease, and ii) a crRNA capable of directing sequence-specific binding of the Cpf1 endonuclease to the first DNA fragment.
   4. The method of embodiment 3, wherein the Cpf1 endonuclease is non-naturally occurring.
   4.1 The method of embodiment 4, wherein the Cpf1 endonuclease is translationally fused to a ligase via a linker sequence.
   4.2 The method of embodiment 4 or 4.1, wherein the Cpf1 endonuclease comprises a nuclear localization signal (NLS).
   5. The method of any one of embodiments 3 or 4.2, wherein the crRNA is non-naturally occurring.
   6. The method of any one of embodiments 1-5, wherein the Cpf1 CRISPR complex of step (b) is targeted to a portion of the first DNA fragment that will be cleaved away from the first DNA fragment, such that the Cpf1 CRISPR complex no longer targets the digested first DNA fragment.
   7. The method of embodiment 6, wherein the Cpf1 CRISPR complex is targeted to a portion of the first DNA fragment that will result in the creation of a sticky end corresponding to the sequence overlap between the first DNA fragment and the second DNA fragment.
   8. The method of any one of embodiments 1-7, wherein steps (b), and (d) are conducted in the same reaction without needing to inactivate the Cpf1 CRISPR complex.
   9. The method of any one of embodiments 1-8, wherein the provided second DNA fragment comprises a preexisting sticky end compatible with the sticky end of the digested first DNA fragment.
   10. The method of any one of embodiments 1-9, wherein step (b) further comprises digesting the second DNA fragment with a second Cpf1 CRISPR complex, thereby creating a second sticky DNA end at the 5′ and/or 3′ of said second DNA fragment, wherein said digested second DNA fragment ceases to be a target for said second Cpf1 CRISPR endonuclease system.
   11. The method of embodiment 10, wherein the first Cpf1 CRISPR complex and the second Cpf1 CRISPR complex are identical (e.g., use the same crRNA).
   12. A method for editing the genome of a cell in vivo, said method comprising the steps of:
a) introducing into the cell one or more vectors encoding for at least two Cpf1 CRISPR complexes, said one or more vectors comprising:
   i) a first polynucleotide encoding for a first crRNA that hybridizes to a first selected target sequence within the genome of the cell;
   ii) a second polynucleotide encoding a second crRNA that hybridizes to a second selected target sequence within the genome of the cell; and
   iii) a third polynucleotide encoding a Cpf1 endonuclease;
wherein components (i), (ii), and (iii) are expressed in the cell, and the Cpf1 endonuclease cleaves the cell's genome at the first and second selected target sequences, thereby producing sticky ends on the cleaved ends of the cell's genome;
wherein the first and second target sequences are positioned in an outwardly facing inverse orientation of a portion of the cell's genome slated for removal, such that removal of said portion of the cell's genome will also remove the first and second target sites from the genome;
(b) annealing the resulting genome sticky ends to each other; and
(c) ligating the annealed genome sticky ends from step (b).
   13. The method of embodiment 12, wherein the one or more vectors of step (a) further comprise a fourth, insert polynucleotide, wherein said insert polynucleotide is also cleaved by the Cpf1 endonuclease, thereby creating sticky ends on the insert polynucleotide that are compatible with the sticky ends of the cell's genome;
   wherein the annealing step (b) is modified to anneal the sticky ends of the genome to the sticky ends of the insert polynucleotide; and
   wherein the ligating step (c) is modified to ligate the annealed genome and insert sticky ends.
   14. The method of embodiment 12 or 13, wherein no genetic scars are introduced into the genome from practicing the method.
   15. The method of embodiment 13, wherein the fourth, insert polynucleotide, also comprises two copies of the first target sequence positioned in an inwardly facing inverse orientation, such that cleavage of said insert polynucleotide by the Cpf1 endonuclease removes the first and second copies of the first target site from the insert polynucleotide.
   16. The method of any one of embodiments 12-15, wherein the one or more vectors comprise a fifth polynucleotide, said fifth polynucleotide encoding a DNA ligase.
   17. The method of embodiment 16, wherein the DNA ligase is selected from the group consisting of T4 ligase, and a T7 ligase.
   18. The method of any one of embodiments 12-17, wherein the Cpf1 endonuclease is non-naturally occurring.
   18.1 The method of embodiment 18, wherein the Cpf1 endonuclease is translationally fused to a ligase via a linker sequence.
   18.2 The method of embodiment 18 or 18.1, wherein the Cpf1 endonuclease comprises a nuclear localization signal (NLS).
   19. The method of any one of embodiments 12-18.2, wherein the first or second crRNA is non-naturally occurring.
   20. The method of any one of embodiments 16, and 18-19, wherein the DNA ligase is non-naturally occurring.
   21. The method of any one of embodiments 12-20, wherein the combination of (i), (ii), and (iii) is non-naturally occurring.
   22. A method for removing a transposon from the genome of a cell in vivo, said method comprising the steps of:
a) introducing into the cell a CRISPR complex encoded in one or more vectors comprising:
   i) a first polynucleotide encoding a first crRNA that hybridizes to a first selected target sequence within the transposon;
   ii) a second polynucleotide encoding a second crRNA that hybridizes to a second selected target sequence within the transposon; and
   iii) a third polynucleotide encoding a CRISPR endonuclease;
wherein components (i), (ii), and (iii) are expressed in the cell, and the CRISPR endonuclease cleaves the cell's genome at the first and second selected target sequences, thereby producing sticky ends on the cleaved ends of the cell's genome;
wherein the first and second target sequences are positioned in an outwardly facing inverse orientation of within the transposon, such that removal of said transposon will also remove the first and second target sites from that portion of the genome;
(b) annealing the resulting genome sticky ends to each other; and
(c) ligating the annealed genome sticky ends from step (b), resulting in a ligated genome; wherein the resulting ligated genome lacks said transposon.
   23. The method of embodiment 22, wherein no genetic scars are introduced into the genome from practicing the method.
   24. The method of embodiment 22 or 23, wherein the one or more vectors further comprise a fifth polynucleotide, said fifth polynucleotide encoding a DNA ligase.
   25. The method of embodiment 24, wherein the DNA ligase is selected from the group consisting of T4 ligase, and a T7 ligase.
   26. The method of any one of embodiments 22-25, wherein the CRISPR endonuclease is non-naturally occurring.
   26.1 The method of embodiment 26, wherein the CRISPR endonuclease is translationally fused to a ligase via a linker sequence.
   26.2 The method of embodiment 26 or 26.1, wherein the CRISPR endonuclease comprises a nuclear localization signal (NLS).
   27. The method of any one of embodiments 22-26.2, wherein the first or second crRNA is non-naturally occurring.
   28. The method of any one of embodiments 24, and 26-27, wherein the DNA ligase is non-naturally occurring.
   29. The method of any one of embodiments 22-28, wherein the combination of (i), (ii), and (iii) is non-naturally occurring.
   30. The method of any one of embodiments 22-29, wherein the CRISPR endonuclease is Cpf1.
   31. A method for assembling gene constructs in vitro from a plurality of DNA fragments, said method comprising the steps of:
(a) providing a plurality of DNA fragments comprising a first and second DNA fragment, wherein said first DNA fragment comprises a sequence overlap of at least three nucleic acids anywhere within the second DNA fragment;
(b) digesting the first DNA fragment with a Targetable Enzyme, thereby creating a sticky DNA end at the 5′ and/or 3′ of said first DNA fragment, wherein said digested first DNA fragment ceases to be a target for said Targetable Enzyme;
(c) annealing the sticky end of the digested first DNA fragment from step (b) to a second compatible sticky end on the second DNA fragment; and
(d) ligating the annealed DNA fragments from step (c) together, resulting in a ligated product;
   wherein the resulting ligated product is an assembled construct.
   32. The method of embodiment 31, wherein no genetic scars are introduced into the assembled construct from practicing the method.
   33. The method of embodiment 31 or 32, wherein the Targetable Enzyme comprises a Cas9 endonuclease translationally fused to a DNA nuclease capable of producing 5′ or 3′ overhangs.
   34 The method of embodiment 33, wherein the Cas9 is translationally fused to the DNA nuclease via a linker sequence.
   35 The method of embodiment 33 or 34, wherein the Cas9 comprises a nuclear localization signal (NLS).

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not, be taken as an acknowledgment or any form of suggestion that they constitute valid prior art or form part of the common general knowledge in any country in the world.

1. A method for assembling gene constructs in vitro from a plurality of DNA fragments, said method comprising the steps of:
(a) providing a plurality of DNA fragments comprising a first and second DNA fragment, wherein said first DNA fragment comprises a sequence overlap of at least three nucleic acids anywhere within the second DNA fragment;
(b) digesting the first DNA fragment with a Cpf1 CRISPR system, thereby creating a sticky DNA end at the 5′ and/or 3′ of said first DNA fragment, wherein said digested first DNA fragment ceases to be a target for said Cpf1 CRISPR system;
(c) annealing the sticky end of the digested first DNA fragment from step (b) to a second compatible sticky end on the second DNA fragment; and
(d) ligating the annealed DNA fragments from step (c) together, resulting in a ligated product;
wherein the resulting ligated product is an assembled construct;
wherein steps (b) and (d) are conducted in the same reaction without needing to inactivate the Cpf1 CRISPR system.

2. The method of claim 1, wherein no genetic scars are introduced into the assembled construct from practicing the method.

3. The method of claim 1, wherein the Cpf1 CRISPR system comprises i) a Cpf1 endonuclease, and ii) a crRNA capable of directing sequence-specific binding of the Cpf1 endonuclease to the first DNA fragment.

4. The method of claim 3, wherein the Cpf1 endonuclease is non-naturally occurring.

5. The method of claim 3, wherein the crRNA is non-naturally occurring.

6. The method of claim 1, wherein the Cpf1 CRISPR system of step (b) is targeted to a portion of the first DNA fragment that will be cleaved away from the first DNA fragment, such that the Cpf1 CRISPR system no longer targets the digested first DNA fragment.

7.-8. (canceled)

9. The method of claim 1, wherein the provided second DNA fragment comprises a preexisting sticky end compatible with the sticky end of the digested first DNA fragment.

10. The method of claim 1, wherein step (b) further comprises digesting the second DNA fragment with a second Cpf1 CRISPR system, thereby creating the second compatible sticky end on the second DNA fragment, wherein said digested second DNA fragment ceases to be a target for said second Cpf1 CRISPR endonuclease system.

11. The method of claim 10, wherein the first Cpf1 CRISPR system and the second Cpf1 CRISPR system are identical.

12. A method for editing the genome of a cell in vivo, said method comprising the steps of:
a) introducing into the cell a Cpf1 CRISPR system comprising one or more vectors comprising:
i) a first polynucleotide encoding a first crRNA that hybridizes to a first selected target sequence within the genome of the cell;
ii) a second polynucleotide encoding a second crRNA that hybridizes to a second selected target sequence within the genome of the cell; and
iii) a third polynucleotide encoding a Cpf1 endonuclease;
wherein components (i), (ii), and (iii) are expressed in the cell, and the Cpf1 endonuclease cleaves the cell's genome at the first and second selected target sequences, thereby producing sticky ends on the cleaved ends of the cell's genome;
wherein the first and second target sequences are positioned in an outwardly facing inverse orientation of a portion of the cell's genome slated for removal, such that removal of said portion of the cell's genome will also remove the first and second target sites from the genome;
(b) annealing the resulting genome sticky ends to each other; and
(c) ligating the annealed genome sticky ends from step (b).

13. The method of claim 12, wherein the one or more vectors of step (a) further comprise a fourth, insert polynucleotide, wherein said insert polynucleotide is also cleaved by the Cpf1 endonuclease, thereby creating sticky ends on the insert polynucleotide that are compatible with the sticky ends of the cell's genome;
wherein the annealing step (b) is modified to anneal the sticky ends of the genome to the sticky ends of the insert polynucleotide; and
wherein the ligating step (c) is modified to ligate the annealed genome and insert sticky ends.

14. The method of claim 12, wherein no genetic scars are introduced into the genome from practicing the method.

15. The method of claim 13, wherein the fourth, insert polynucleotide, also comprises two copies of the first target sequence positioned in an inwardly facing inverse orientation, such that cleavage of said insert polynucleotide by the Cpf1 endonuclease removes the first and second copies of the first target site from the insert polynucleotide.

16. The method of claim 12, wherein the one or more vectors comprise a polynucleotide encoding a DNA ligase.

17. The method of claim 16, wherein the DNA ligase is selected from the group consisting of T4 ligase, and a T7 ligase.

18. The method of claim 12, wherein the Cpf1 endonuclease is non-naturally occurring.

19. The method of claim 12, wherein the first or second crRNA is non-naturally occurring.

20. The method of claim 16, wherein the DNA ligase is non-naturally occurring.

21. The method of claim 12, wherein the combination of (i), (ii), and (iii) is non-naturally occurring.

22.-30. (canceled)

31. The method of claim 13, wherein no genetic scars are introduced into the genome from practicing the method.
